(19) United States
(12) Patent Application Publication
(10) Pub. No.: US 2016/0244847 A1
(43) Pub. Date: Aug. 25, 2016

(54) PREDICTING INCREASED RISK FOR CANCER

(71) Applicant: CRAY INNOVATION AB, Uppsala (SE)

(72) Inventors: Lars Forsberg, Uppsala (SE); Jan Dumanski, Uppsala (SE)

(73) Assignee: Cray Innovation AB, Uppsala (SE)

(21) Appl. No.: 15/028,012

(22) PCT Filed: Oct. 7, 2014
(86) PCT No.: PCT/EP2014/071448

§ 371 Date: Apr. 7, 2016

(30) Foreign Application Priority Data
Oct. 7, 2013 (SE) 1351178-7

(51) Int. Cl. C12Q 1/68

(52) U.S. Cl. CPC C12Q 1/6886; C12Q 2600/156

(57) ABSTRACT

Male subjects having an increased risk of developing cancer are identified by determining a fraction of cells that have lost chromosome Y in a biological sample obtained from the male subject. The fraction is compared to a predefined threshold and the male subject is predicted to have an increased risk of developing cancer based on the comparison between the fraction and the predefined threshold.

### TECHNICAL FIELD

The present embodiments generally relate to predicting increased risk of developing cancer among male subjects using a genetic marker.

### BACKGROUND

There currently exist many methods for prediction of risks for various diseases. An example from the non-cancer-related field might be an increased blood pressure, which is associated with higher risk of stroke and cardiovascular disease. Other examples related to cancer are screening using breast mammography for detection of breast cancer and radiology of the lung for detection of asymptomatic lung cancers. However, the latter two methods detect disease processes that have already progressed to radiologically detectable tumors.

The field of human genetics has also generated a number of methods, which are useful in early prediction of risk for various diseases and these methods can be broadly divided into three categories. Cytogenetic analyses were the first allowing discovery of human mutations (changes on a chromosomal level or large sub-chromosomal aberrations), which allowed calculation of the risk of development of a specific disease phenotype, usually in the context of prenatal diagnostics. For instance, trisomy 21, which is connected with a strong risk for the development of Down syndrome, is a classic example. This and other various cytogenetically detectable chromosomal aberrations usually originate from errors occurring in meiosis of one of the parents and lead to aberrant phenotype in developing embryos or children.

The second large genetic field is concerning monogenic disorders. During the last two decades of the past century, the field of human genetics was to a large extent dominated by studies of monogenic disorders, leading to cloning of a very large number of genes implicated in disease development. Mutations in the genes causing monogenic disorders were usually either inherited from an affected parent or were occurring as new mutations during meiosis leading to a gamete carrying a disease mutation. The importance of these methods is also mainly in the context of prenatal diagnostics and the general medical impact is limited due to the fact that the majority of monogenic disorders are usually rare. A few relevant examples might be Huntington's disease and familial breast or colon cancer.

In addition, the completion of sequencing of the human genome and discovery of a frequent inter-individual variation in DNA sequence for single nucleotides, so called single nucleotide polymorphisms (SNPs), has opened another field of investigations in human genetics. These analyses are usually abbreviated as Genome Wide Association Studies (GWAS) and are based on an assumption that nucleotide variants connected with a certain disease risk are either inherited from a parent or they occur very early during embryonic development and are present in DNA of all cells in the body. A very large number of such studies have been published during the past 10 years, dealing with a wide variety of human phenotypes and complex common disorders. However, the predictions of risks associated between nucleotide variants and phenotypes are usually very small and the portion of the estimated disease heritability explained by the GWAS findings has also been unexpectedly low [1].

Thus, in summary for genetic methods of disease risk predictions, during the past 5 decades projects in human genetics investigating correlations between genotype and phenotype have mostly focused on analyses of the inherited genome, which is composed of the variants of genes that were transferred from the parents to the offspring. The prevailing approach has been analysis of DNA from a single tissue (usually blood) that was sampled at a single time point (non-longitudinal sampling). The rationale in these studies has been the assumption that the vast majority of the cells in the human soma are genetically identical; in other words that the genome of somatic cells is stable across the human life span.

Chromosome Y is the human male sex chromosome. It has been known for half a century, first published in 1963, that elderly males frequently lose chromosome Y in normal hematopoietic cells [2, 3]. However, the clinical consequences of this aneuploidy have been unclear and the currently prevailing consensus in the literature suggests that this genetic change should be considered phenotypically neutral and related to normal aging [4-9]. Furthermore, previous studies have described the occurrence of LOY in up to 20 different human malignancies in combination with numerous other aberrations [10-14].

Finally, men show a higher incidence and higher mortality for most sex-unspecific cancers, but this bias is largely unexplained by known risk factors [19, 20].

### SUMMARY

It is a general objective to provide a tool that can be used to identify subjects having a risk of developing cancer.

It is a particular objective to provide a tool that can be used to identify subjects currently not diagnosed for any cancer disease but having an increased risk of developing cancer in the future.

These and other objectives are met by embodiments as defined herein.

An aspect of the embodiments relates to a method of predicting whether a male subject has an increased risk of developing cancer. The method comprises determining a fraction of cells, from a biological sample obtained from the male subject, which have lost chromosome Y. The determined fraction of cells is compared to a predefined threshold. The method also comprises predicting whether the male subject has an increased risk of developing cancer based on the comparison between the fraction of cells and the predefined threshold.

Another aspect of the embodiments relates use of loss of chromosome Y in cells from a biological sample obtained from a male subject as a genetic marker to predict whether the male subject has an increased risk of developing cancer.

A further aspect of the embodiments relates to a kit for predicting whether a male subject has an increased risk of developing cancer. The kit comprises means for determining a fraction of cells, from a biological sample obtained from the male subject, which have lost chromosome Y. The kit also comprises instructions for comparing the determined fraction to a threshold defined by the instructions. The kit further comprises instructions for predicting whether the male subject has increased risk of developing cancer based on the comparison between the determined fraction and the threshold.

### BRIEF DESCRIPTION OF THE DRAWINGS

The embodiments, together with further objects and advantages thereof, may best be understood by making reference to the following description taken together with the accompanying drawings, in which:

FIG. 1—Principles of CRAY-evaluation (Cancer Risk Assessment from LOY-status). This schematic view illustrates the clinical setting and how the CRAY-evaluation can identify subjects at risk to develop cancer. Black box represents an embodiment, with assessment of the degree of loss of chromosome Y (LOY-status) and subsequent prediction of cancer risk in adult/senior/elderly men. In an embodiment, the Illumina SNP-array technology (Single-Nucleotide-Polymorphism-array) is the molecular analysis performed to produce the data that is analyzed in the CRAY-evaluation. In other embodiments, any other genetic (or other) methods such as NGS (Next-Generation-Sequencing), CGH-arrays (Comparative-Genomic-Hybridization), RNA-assays, gene-expression technology, methylome technology, protein-assays, cytogenetic and microscopy methods etc. could be used to produce the data that are used to estimate the LOY-status and/or perform the CRAY-evaluation.

FIG. 2—Estimation of frequency of somatic loss of chromosome Y (LOY) in 1141 elderly men of the ULSAM-cohort. LOY frequency estimation was performed after accounting for experimental variation. Panel A shows the median Log R Ratio (LRR) from SNP-array genotyping in the male specific part of chromosome Y (mLRR-Y) and each triangle represents one participant. Panel B shows the distribution of the mLRR-Y (black bars) and the experimental noise (white bars) that were used to find the threshold for estimation of LOY frequency. The dotted black lines represent the 99% confidence intervals (CI) of the distribution of expected experimental background noise (white bars). Among the analyzed men we found that 14.7% had a lower median LRR than the lower 99% CI representing LOY in ˜13.1% of cells. For estimating the frequency of LOY, we used the lowest value in the noise-distribution as threshold T1 (line at −0.139), which yielded a frequency in the population at 8.2% of men. The threshold T2 at −0.4 was used in survival analyses.

FIG. 3—Estimation of the percentage of blood cells affected with loss of chromosome Y (LOY) through analysis of SNP-array data from the pseudoautosomal region 1 (PAR1) of chromosomes X/Y using MAD-software [16]. PAR1 is the largest of the PARs (regions with homologous sequences on chromosomes X and Y) with coordinates 10001-2649520 on Y and 60001-2699520 on X. MAD-software is a tool for detection and quantification of somatic structural variants from SNP-array data, which uses diploid B-allele frequency (BAF) for identification and Log R Ratio (LRR) for quantification of somatic variants and is not originally intended for analyses of chromosome Y data. However, by using the correlation between the LRR in the PAR1-region of Y and the delta-BAF, i.e. the absolute deviation from the expected BAF-value of 0.5 in heterozygous probes, of the PAR1-region of X/Y (panel A), we could use the MAD-quantification of the diploid PAR1 region on chromosomes X/Y to calculate the percentage of cells affected with LOY (panel B). For example, the delta-BAF-value at the LRR-threshold for survival analyses (mLRR-Y≦0.4) was found using the equation given in panel A, i.e. 0.178. This equation (y=−2.7823x+0.0954) is describing the relationship between mLRR-Y on Y and delta-BAF on X/Y. Next, the percentage of cells affected by LOY was found by applying the equation in panel B that describes the relationship between delta-BAF and the percentage of cells as estimated by the MAD software for 14 cases (y=1.832x+0.023). For this example, the delta-BAF of 0.178 translates to LOY in 35% of cells.

FIG. 4—Illustration of longitudinal changes in six ULSAM-participants in panels A-F. For each subject, the Log R Ratio from SNP-array analyses of chromosome Y is displayed from three different ages. The normal state with no (or un-detectable) loss of chromosome Y (LOY) is found close to zero. For example, in panel A, the subject ULSAM 102 at 78 years old shows a normal profile. Subsequently, at ages 83 and 88, the fraction of cells with LOY increases in the blood. A line to indicate the median Log R Ratio of all SNP-probes in the male specific part of chromosome Y (mLRR-Y) is plotted to indicate the level of LOY. Panels B through F show similar patterns of progressive accumulation of cells containing LOY with increasing age in subjects 835, 942, 1074, 1412 and 1435, respectively. These six cases sampled and analyzed in longitudinal fashion shows a typical pattern with a clear increase of the fraction of cells with LOY in blood. This is a strong indication that the affected cells have a proliferative advantage as compared to wildtype cells with unaffected LOY-status.

FIG. 5—LOY and its effect on mortality in the ULSAM cohort. Panels A, B and C show impact of LOY on all-cause mortality, cancer mortality and mortality from non-hematological cancers, respectively, in 982 men with no history of cancer prior to sampling. Hazard ratios (HR), 95% confidence intervals (CI), number of events and p-values are shown for each model. Results are derived from Cox proportional hazards regression models, with subjects classified into groups 1 and 0 based on their level of LOY. Individuals in the affected group (lower curve in each panel) had LOY in ≧35% of nucleated blood cells (see FIG. 2).

FIG. 6—Results from exploratory survival analyses using Cox proportional hazards regression models with different thresholds for classification of participants into groups 1 and 0, based on their level of loss of chromosome Y (LOY) measured as the median Log R Ratio (LRR) in the male specific part of chromosome Y (mLRR-Y). The number of participants (n) with LOY and the minimum percentage of affected cells for each subject are given for each of the tested thresholds. The upper and lower curves represent results from analyses with cancer mortality or all-cause mortality as endpoints, respectively. Based on these results from ULSAM cohort, mLRR-Y at −0.4 is the most informative threshold for survival analyses in the studied cohort.

FIG. 7—Example 1 shows a patient called ULSAM-311 who was genotyped on SNP-array at two different ages, 75 and 88 years old, and he subsequently died from prostate cancer at the age of 89. Somatic loss of chromosome Y (LOY) was present at both genotyping ages with 37% and 74% of cells affected, respectively. A CRAY-evaluation at both of these ages would have predicted a great risk of cancer. For example, an oncological evaluation at 75 (initiated by a CRAY-evaluation performed at the 75 years DNA) could have resulted in a three years earlier diagnosis of the prostate cancer that subsequently led to the death of this man. Screening of the blood at younger ages would probably entail an even earlier diagnosis.

FIG. 8—Example 2 shows a subject ULSAM-41 who was genotyped using SNP-array at two ages, 83 and 88 years old, and died from an un-diagnosed cancer at the age of 90. Somatic loss of chromosome Y (LOY) was present at both genotyping ages with 20% and 45% of cells affected, respectively. A CRAY-evaluation at any of these ages would have predicted an increased risk of cancer and subsequently a great risk of cancer. The following oncological examination could have detected the cancer that caused the death of this man seven years later. For example, a CRAY-evaluation using the data from the sample collected at 83 years of age would have predicted an increased risk for cancer and a better treatment for this man. However, the cancer that caused the mortality was only discovered post mortem.

FIG. 9—Example 3 showing a subject ULSAM-33 who was genotyped on SNP-array at two ages, 72 and 83 years old, and died from prostate cancer at the age of 87. Somatic loss of chromosome Y (LOY) was present at both genotyping ages with 27% and 41% of cells affected, respectively. A CRAY-evaluation at both of these ages would have predicted an increased cancer risk and subsequently a great risk of cancer. For example, an oncological evaluation at 72 (initiated by a CRAY-evaluation performed at the 72 years DNA) could have resulted in a four years earlier diagnosis of the prostate cancer that subsequently led to his death. Screening of blood sampled at even younger ages would probably entail an even earlier diagnosis.

FIG. 10—Example 4 illustrating the potential benefits of CRAY-evaluation using a hypothetical cancer patient in two different scenarios, depicted in panels A and B. In the first scenario shown in panel A, no CRAY-evaluation is performed and the subject dies from a cancer at the age of 75. This scenario is very similar to examples 1-3 (in FIGS. 7-9) and the clinical reality of contemporary cancer screening. In the alternative scenario shown in panel B, the CRAY-evaluation performed at the age of 65 detects the risk factor for cancer. In this patient an oncological evaluation could diagnose a cancer that was treated in a very early phase of carcinogenesis.

FIG. 11—A flow diagram illustrating method of predicting whether a male subject has an increased risk of developing cancer according to an embodiment.

FIG. 12—A flow diagram illustrating an additional, optional step of the method as shown in FIG. 11.

### DETAILED DESCRIPTION

The present embodiments generally relate to predicting increased risk of developing cancer among male subjects using a genetic marker.

The present embodiments are related to a field of human genetics generally denoted “somatic mosaicism” or “post-zygotic mosaicism”. These terms define the presence of genetically distinct lineages of cells within a single organism, which is derived from the same zygote. In other words, they refer to DNA changes acquired during lifetime, ranging in size from single base pair mutations to aberrations at the chromosomal level [1].

In more detail, the present embodiments are based on the unexpected discovery that loss of human chromosome Y in normal cells, such as nucleated blood cells, of adult and elderly men, is associated with earlier mortality and increased risk for cancer, n particular various forms of non-hematological cancers.

In brief, there is, which is further shown herein, a strong association between somatic loss of chromosome Y (LOY) in cells and risk for developing cancer. This opens up for the prediction of individual cancer risks.

Previous studies have described the occurrence of somatic loss of chromosome Y in normal haematopoetic cells, but were unable to describe the link between this aberration and risk for mortality and cancer. Furthermore, somatic loss of chromosome Y has been described in up to 20 different human malignancies in combination with numerous other aberrations [10-14]. These latter studies, thus, show that some cancer types are associated with chromosome abnormalities or aberrations in the tumor cells in terms of LOY. However, there is no indication in these studies that LOY in normal blood cells could be used for predicting an increased risk for future cancer in still healthy individuals.

Thus, the present embodiments provide a tool that can be used to test or investigate male subjects that currently are not diagnosed for cancer and are thereby regarded as being healthy at least in terms of not having any diagnosed cancer disease. The tool is able to identify those healthy individuals that have an increased risk of developing cancer and, consequently, have a risk of suffering from cancer in the future.

The present embodiments can, thus, be used to screen for and identify male subjects that have an increased risk, compared to male subjects in general, of developing cancer as predicted based on LOY as disclosed herein.

Increased risk should thereby be regarded herein as a risk level of developing cancer that is elevated and thereby higher than any general risk among male subjects of developing cancer. The increased risk can be seen as a risk in addition to and above any normal or baseline risk of developing cancer that exist among all male subjects.

The present embodiments thereby do not provide any cancer diagnosis since the identified male subjects are currently not suffering from any cancer disease but rather enable identification of those male subjects that have a comparatively high risk of developing cancer during their remaining lifetime.

Increased risk of developing cancer as predicted based on LOY as disclosed herein implies an increased risk of suffering from cancer in the future as compared to control subjects that do not show any significant detectable loss of chromosome Y.

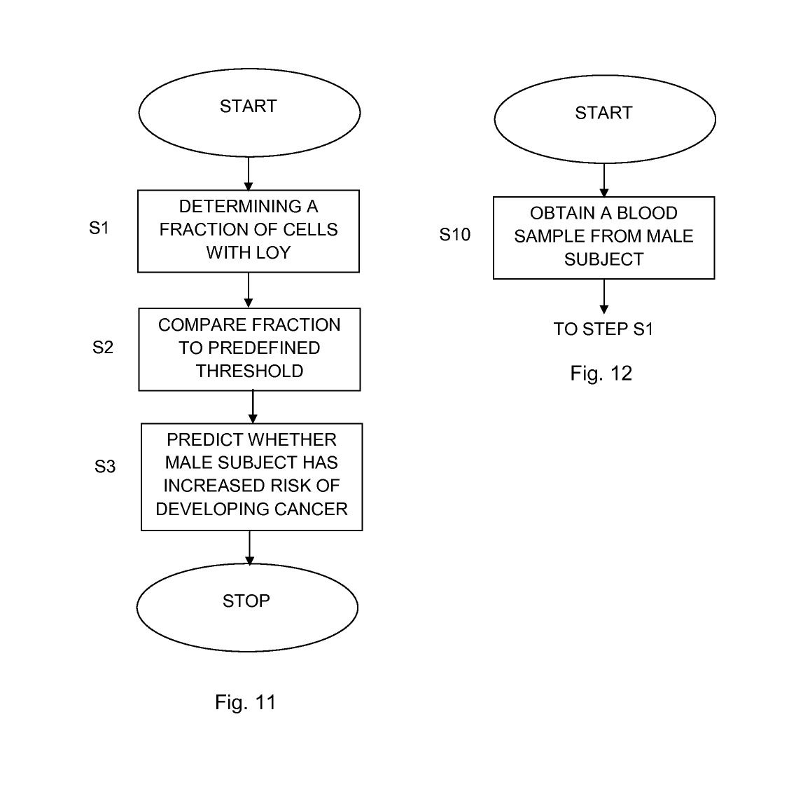

Accordingly, an aspect of the embodiments relates to a method of predicting whether a male (human) subject has an increased risk of developing cancer. The method comprises, as shown in FIG. 11, determining, in step S1, a fraction of cells, from a biological sample obtained from the male subject, that have lost chromosome Y. This fraction is compared to a predefined threshold in step S2. A following step S3 comprises predicting whether the male subject has an increased risk of developing cancer based on the comparison between the fraction and the predefined threshold.

Thus, the embodiments determine the fraction, percentage or degree of cells that have lost chromosome Y in the biological sample taken from the male subject. If this fraction is sufficiently high, as determined in the comparison with the predefined threshold, the male subject is predicted to have an increased risk of developing cancer even if the male subject currently does not have any diagnosed cancer disease.

In a particular embodiment, determining the fraction in step S1 of FIG. 11 comprises determining a fraction of somatic cells from the biological sample that have lost chromosome Y. Thus, in this embodiment the fraction of cells that have lost Y in the biological sample is determined among somatic cells. This means that any gametes, if present in the biological sample, are preferably excluded from the determination of the fraction of cells.

The present embodiments can generally be used to predict an increased risk of developing cancer, and in particular non-hematological cancer. Hence, the embodiments are in particular suitable for predicting whether the male subject has an increased risk of developing non-hematological cancer based on the comparison between the fraction determined in step S1 of FIG. 11 and the predefined threshold.

The biological sample as obtained from the male subject can be any biological sample that comprises cells, preferably somatic cells, of the male subject. Non-limiting but suitable examples of biological sample include a blood sample, a lymph sample, a saliva sample, a urine sample, a feces sample, a cerebrospinal fluid sample, a skin sample, a hair sample or indeed any other tissue sample or type of biopsy material taken from the male subject.

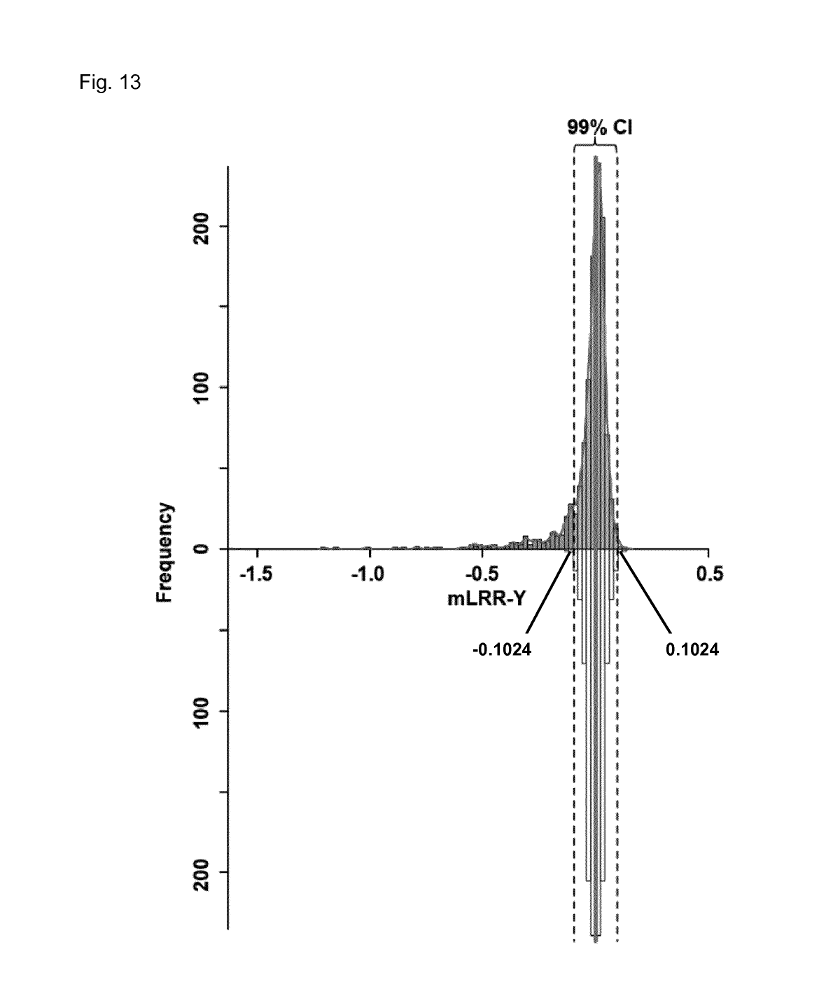

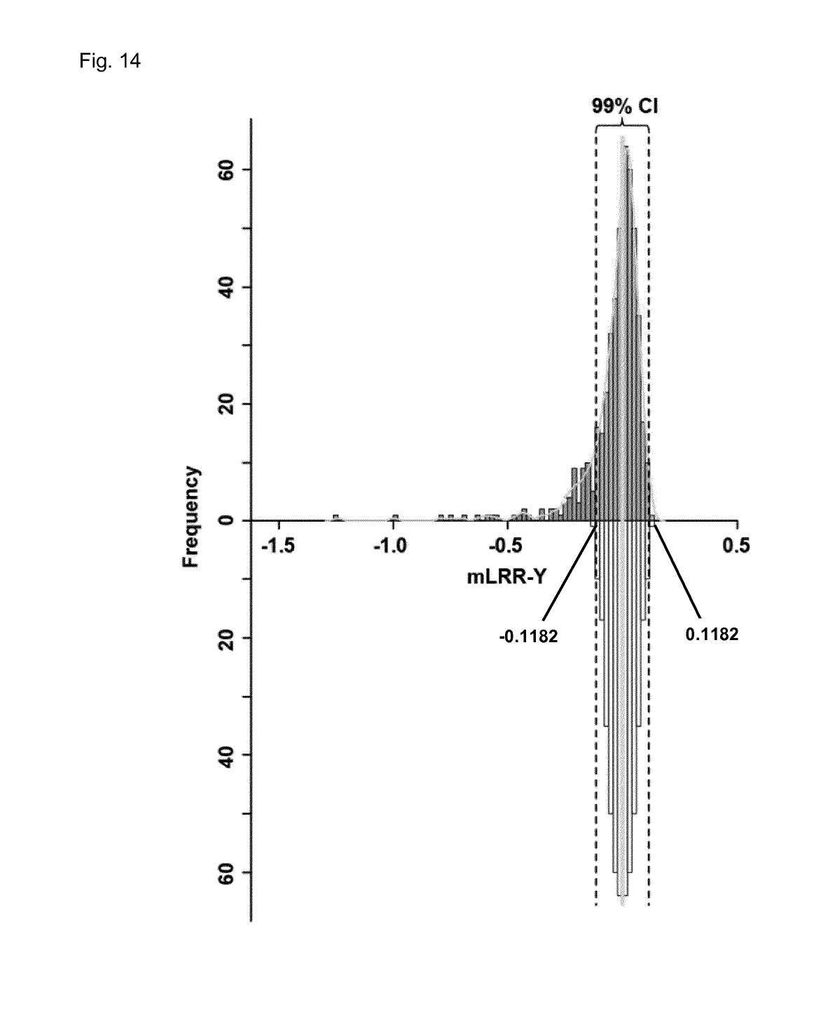

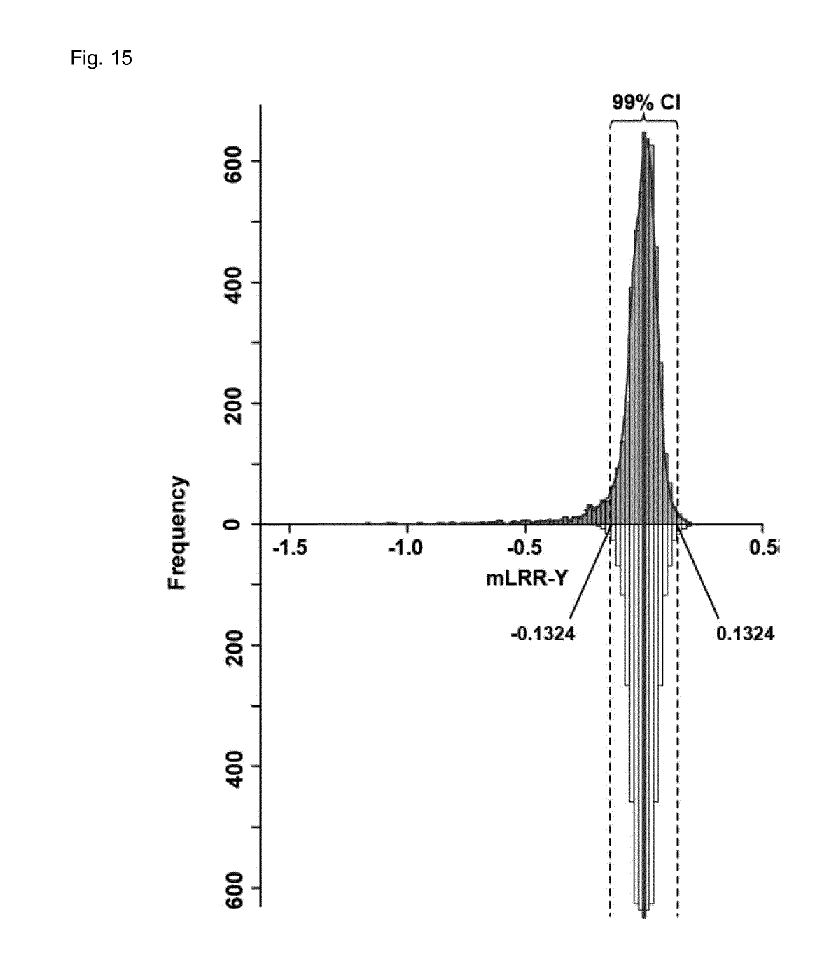

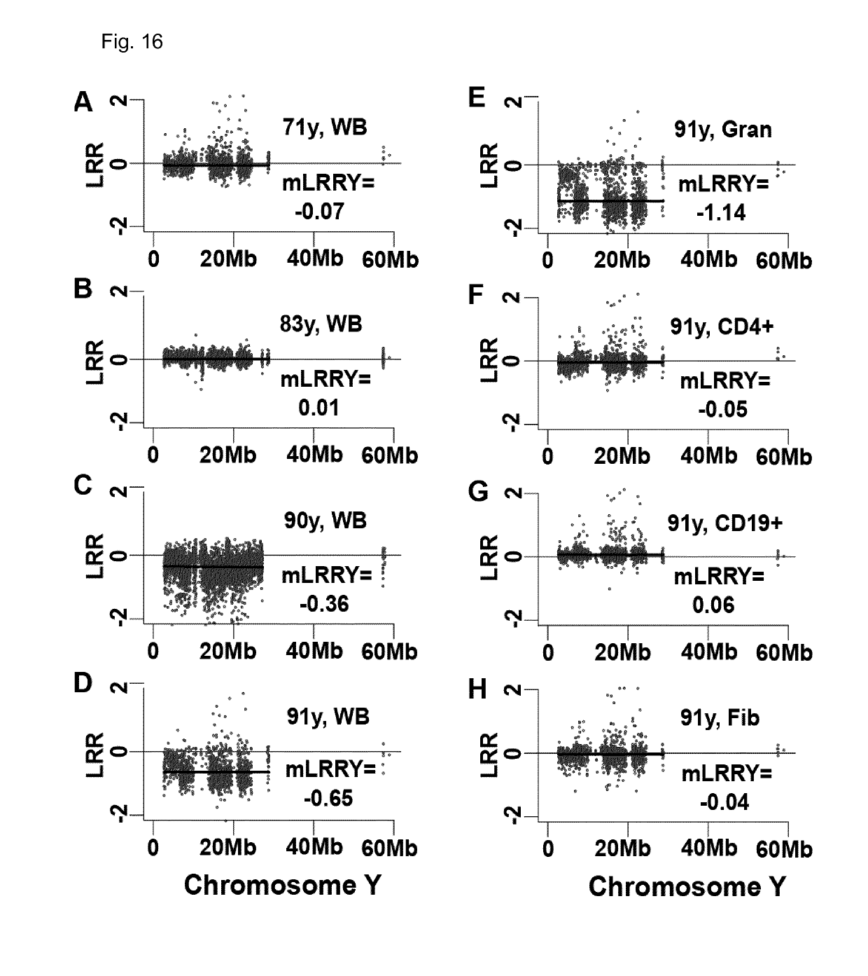

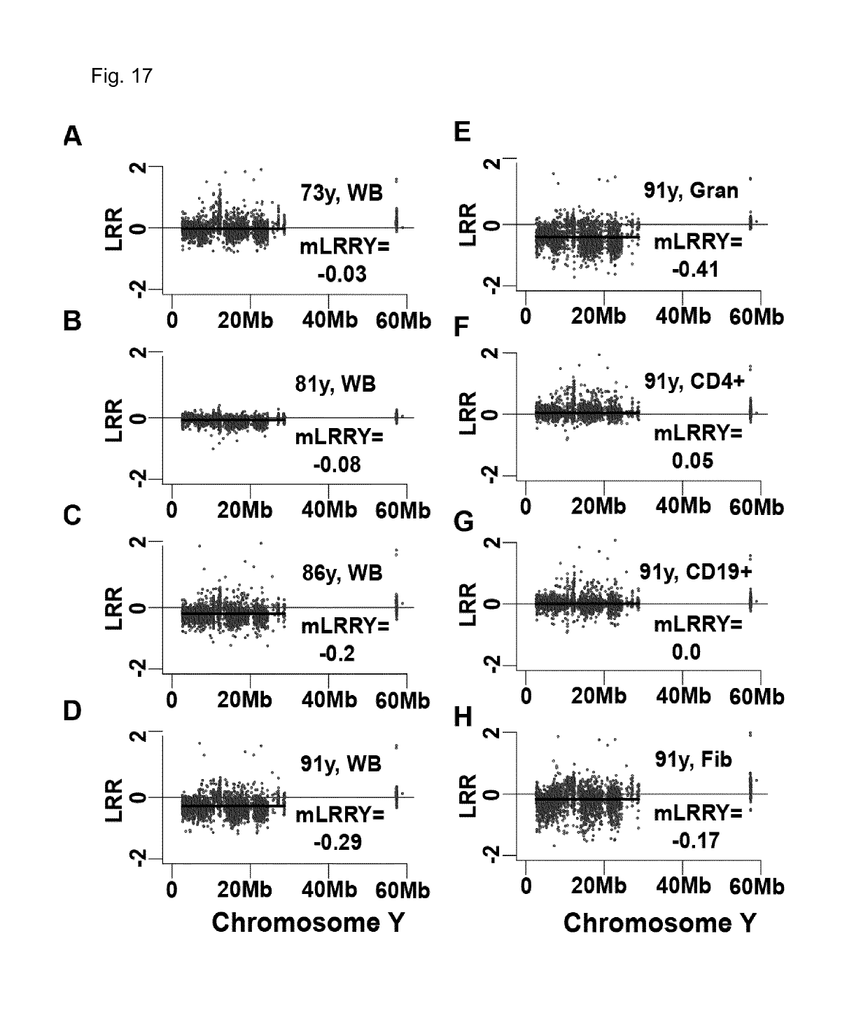

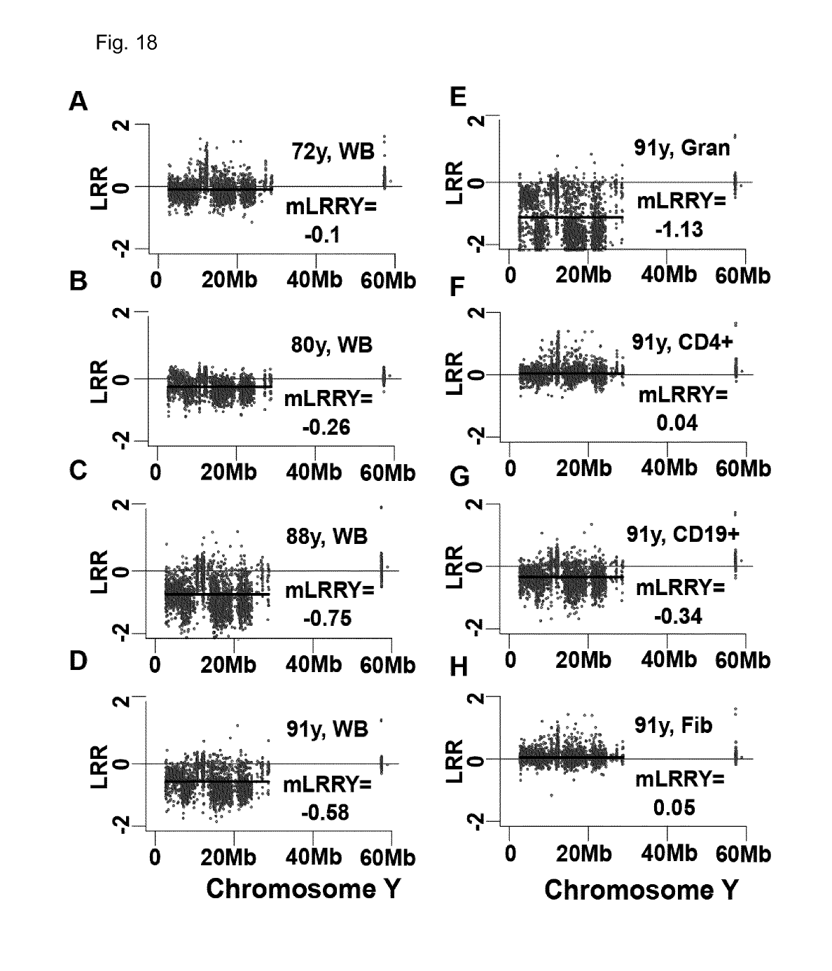

In a particular embodiment, the biological sample is a blood sample. Accordingly, the method as shown in FIG. 11 then preferably comprises an additional step of obtaining a blood sample from the male subject in step S10 as shown in FIG. 12. The determination of the fraction of cells in step S1 of FIG. 11 then preferably comprises determining the fraction of nucleated blood cells from the blood sample that have lost chromosome Y.

As is well known in the art, a blood sample comprises various blood cells, and in particular red blood cells, i.e. erythrocytes; white blood cells, i.e. leukocytes; and platelets, i.e. thrombocytes.

Generally, red blood cells and platelets lack a cell nucleus in humans. Accordingly, these blood cells are therefore regarded as non-nucleated blood cells. The determination of the fraction of cells in step S1 preferably involves, in this embodiment, determining the fraction of nucleated blood cells that have lost chromosome Y. This means that in preferred embodiment loss of chromosome Y is investigated in white blood cells in the blood sample since the white blood cells, in clear contrast to the red blood cells and the platelets, contain a cell nucleus. Thus, step S1 of FIG. 11 then preferably comprises determining the fraction of leukocytes from the blood sample that have lost chromosome Y.

In a particular embodiment, the prediction of step S3 comprises predicting that the male subject has an increased risk of developing cancer if the fraction determined in step S1 is equal to or higher than the predefined threshold. Correspondingly, this step S3 preferably comprises predicting that the male subject does not have any increased risk of developing cancer if the fraction is lower than the predefined threshold.

The predefined threshold is thereby employed, in this embodiment, to differentiate those male subjects that do not have increased risk of developing cancer from those that have an increased risk of developing cancer as assessed based on the fraction of the cells that have lost chromosome Y.

The predefined threshold, to which the determined fraction of cells is compared in step S2, can be determined by the person skilled in the art based on determining the fraction of cells that have lost chromosome Y in multiple male subjects that currently are diagnosed to be healthy in terms of not having any diagnosed cancer disease. These male subjects can then be grouped into two main groups: 1) those that later on develop cancer and are thereby later on diagnosed as suffering from cancer and 2) those that never will develop cancer. The predefined threshold can then be set to discriminate between these two main groups.

A non-limiting but illustrative value of the predefined threshold is about 18%. This value was determined as disclosed herein based on analysis of post-zygotic genetic aberrations in a large cohort from Uppsala Longitudinal Study of Adult Male (ULSAM).

It is, however, expected that the predefined threshold used in the comparison in step S2 could typically be selected in an interval from about 5% to about 20%. This means that if at least 5% to 20% of the cells from the biological sample have lost chromosome Y then the male subject is predicted to have an increased risk of developing cancer.

In a particular embodiment, the fraction of cells as determined in step S1 in FIG. 11 is preferably compared, in step S2, to a first predefined threshold and a second predefined threshold that is higher than the first predefined threshold. In such a case, the prediction as conducted in step S3 preferably comprises predicting that the male subject has an increased or moderate risk of developing cancer if the determined fraction is equal to or larger than the first predefined threshold but lower than the second predefined threshold. Step S3 preferably also comprises predicting that the male subject has a high risk of developing cancer if the fraction is equal to or larger than the second predefined threshold. Correspondingly, step S3 preferably also comprises predicting that the male subject does not have any increased risk of developing cancer if the fraction is lower than the first predefined threshold.

In this embodiment, the male subjects are differentiated into three groups: 1) those that do not have any increased risk of developing cancer, 2) those that have an increased but moderate risk of developing cancer, and 3) those that have a high or very high risk of developing cancer.

Thus, there seems to be a relationship between the fraction of cells that have lost chromosome Y and the likelihood or risk of developing cancer. This means that the higher fraction of cells with LOY the higher the risk of developing cancer.

The first and second predefined thresholds can be determined as previously discussed herein. Experimental data from ULSAM indicate that suitable, but non-limiting, values of the first and second predefined thresholds could be 18% and 35%, respectively.

In an embodiment, the second predefined threshold is about twice the value of the first predefined threshold.

This concept can of course be extended to a case using more than two thresholds to differentiate male subjects into more than three different groups.

The determination of the fraction of cells that have lost chromosome Y can be performed according to various analysis methods and techniques. Generally, DNA and/or RNA from the cells in the biological sample is analyzed in order to determine presence or absence of chromosome Y in the cells. For instance, polymerase chain reaction (PCR) primers can be used to detect the presence or absence of any DNA sequence in chromosome Y and where the absence of the chromosome-Y-specific DNA sequence is regarded as LOY. Correspondingly, techniques can be used to detect presence or absence of mRNA transcribed from active genes present on chromosome Y. Absence of any significant detectable amounts of mRNA could then be regarded as LOY. Alternatively, the detection of LOY can be done on protein basis instead of or as an alternative to DNA and/or RNA analysis. In such a case, the presence or absence of a protein encoded by a gene on chromosome Y can be used to discriminate between those cells that have an intact chromosome Y and those cells that have lost chromosome Y.

Further non-limiting examples of analysis methods that can be used include single-nucleotide polymorphism (SNP) array analysis, RNA-assays, gene expression analysis, protein assays, epigenetic assays and various genetic methods, such as sequencing, comparative genomic hybridization (CGH) arrays, methylome assays and cytogenic methods. It is also possible to visually detect LOY using microscopy methods.

Sequencing methods that can be used include basic and advanced sequencing methods, such as Maxam-Gillbert sequencing, Chain-termination methods, Shotgun sequencing and bridge PCT. Also, or alternatively, next-generation sequencing methods could be used to detect LOY and determine the fraction of cells that have lost chromosome Y. Examples of such next-generation sequencing methods include massively parallel signature sequencing (MPSS), polony sequencing, 454 pyrosequencing, Illumina sequencing, SOLID sequencing, Ion Torrent semiconductor sequencing, DNA nanoball sequencing, Heliscope single molecule sequencing, single molecule real time (SMRT) sequencing, nanopore DNA sequencing, tunneling currents DNS sequencing, sequencing by hybridization, sequencing with mass spectrometry, microfluidic Sanger sequencing, microscopy-based techniques, RNAP sequencing and in vitro virus high-throughput sequencing.

In a particular embodiment, the determination of the fraction in step S1 of FIG. 11 is based on SNP array analysis using data points derived from a pseudoautosomal region 1 (PAR1) region of chromosomes X and Y. In particular, the fraction may be determined based on a quantification of the data points derived from the PAR1 region by using a correlation between a Log R Ratio (LRR) in the PAR1 region of chromosome Y and an absolute deviation from an expected diploid B-allele frequency (BAF) value of 0.5 as further disclosed herein.

In another particular embodiment, the determination of the fraction in step S1 is based on sequencing of a DNA region present on chromosome Y.

Another aspect of the embodiments relates to the use of loss of chromosome Y (LOY) in cells from a biological sample obtained from a male subject as a genetic marker to predict whether the male subject has an increased risk of developing cancer.

Thus, in these embodiments LOY is used as a genetic marker for cancer prediction.

The above described method could be performed using a kit. Accordingly, a further aspect of the embodiments relates to a kit for predicting whether a male subject has an increased risk of developing cancer. The kit comprises means for determining a fraction of cells from a biological sample obtained from the male subject that have lost chromosome Y. The kit also comprises instructions for comparing the fraction obtained from the above mentioned means to a threshold defined or listed in the instructions. The kit further comprises instructions for predicting whether the male subject has an increased risk of developing cancer based on the comparison between the fraction and the predefined threshold.

The means for determining the fraction can include instruments, equipment and/or reagents needed to determine the fraction according to any of the previously mentioned methods or techniques. The instructions of the kit preferably specify the threshold, or the first and second predefined thresholds, and preferably also specify that the fraction should be compared to the threshold(s) and that the male subject is identified as having an increased risk of developing cancer if the determined fraction is equal to or exceeds the threshold. Correspondingly, the instructions could specify that the male subject does not have any increased risk of developing cancer if the determined fraction is lower than the threshold.

Various implementation embodiments and aspects will now be further described here below.

The present embodiments are, in an aspect, directed to a method to predict the risk for, preferably non-hematological, cancer in men. In brief, we have discovered a strong association between somatic loss of chromosome Y (LOY) in, for instance, blood cells and risk for mortality in or running a risk of being diagnosed with non-hematological cancer. This opens up for the prediction of individual cancer risks, herein denoted Cancer Risk Assessment from LOY-status (CRAY).

Previous studies have described the occurrence of LOY in up to 20 different human malignancies in combination with numerous other aberrations [10-14]. In contrast to these studies focusing on aberrations found in tumor cells, the present embodiments will be useful for predicting the risk for future cancer in still healthy individuals.

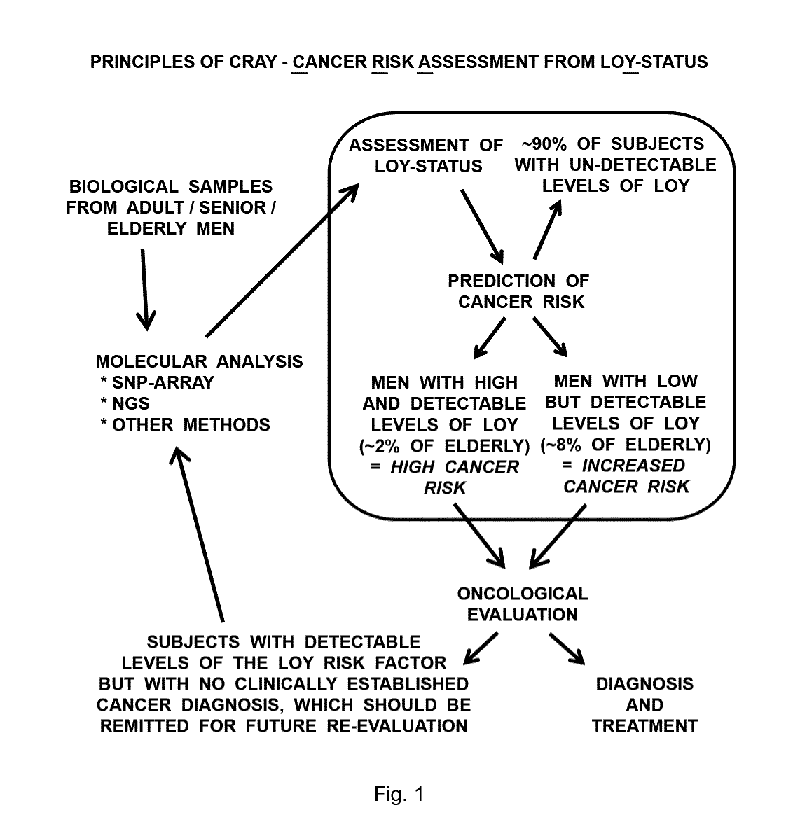

The CRAY-evaluation, indicated by black box in FIG. 1, is based on assessment of LOY-status, achieved by estimation or determination of the fraction, percentage or degree of cells from a biological sample that has lost the chromosome Y. The LOY-status can be performed using genetic analysis, such as SNP-array, next-generation sequencing etc., or with other methodological approaches. When the LOY-status of a subject has been established, the risk for that subject to develop cancer can be assessed by comparing the degree of LOY to pre-defined risk-threshold(s) as described herein. The CRAY-evaluation will, when used in large-scale screening of the general population of adult/senior/elderly men, be able to identify the approximate 10% of elderly men that are at an increased risk for cancer as described below.

In a particular implementation example, a method of determining whether an individual has an increased risk for cancer comprises the steps of:

a) obtaining a biological sample from the individual;

b) determining the fraction, percentage or degree of cells from the biological sample of step a) that has lost the chromosome Y;

c) comparing the result obtained in step b) to pre-defined risk-threshold(s); and

d) determining whether the individual has an increased risk for cancer based on the comparison in step c).

In an embodiment, blood is the tissue type that is sampled and analyzed in the CRAY-evaluation. In other embodiments, the tissue type that is sampled and analyzed may be skin, saliva, feces, urine, cerebrospinal fluid, hair or any other type of biopsy materials.

In an embodiment, senior/elderly men are sampled and analyzed in an analysis using the CRAY-evaluation. In another embodiment, samples are collected at any other ages, such as young or middle-aged subjects/cohorts.

In an embodiment, DNA is analyzed in an analysis using the CRAY-evaluation. In other embodiments, any other sources of biological material or types of molecules are examined to estimate the degree of LOY, such as RNA-assays, gene-expression, protein-assays, and epigenetic-assays etc.

In an embodiment, Illumina SNP-array technology is performed to produce the data that is analyzed in an analysis using the CRAY-evaluation. In other embodiments, any other genetic (or other) methods, such as next-generation sequencing, CGH-arrays, RNA-assays, gene-expression technology, methylome technology, protein-assays, cytogenetic and microscopy methods etc., which can be used to produce the data that, are used to estimate the degree of LOY.

In an embodiment, the assessments of chromosome Y status, i.e. LOY-assessments, are performed using the MAD-software [15] on the SNP-array-data prior to the CRAY-evaluation in an analysis. In other embodiments, also or alternatively any other software or methods to perform the LOY-assessment, such as any methods to determine or estimate the levels of somatic mosaicism, including manual calling, are used.

In an embodiment, the risk of non-hematological cancer is predicted in an analysis using the CRAY-evaluation. In other embodiments, the risks of all cancer types, including both hematological and non-hematological cancers are predicted.

The herein described CRAY-evaluation has numerous potential clinical applications including, but not limited to, prediction of individual cancer risk from LOY-status. Such predictions could be made in large screening programs in the population or in smaller scale during medical check-ups or in personal examination programs. Regardless of the clinical or other settings, the method described will be able to identify subjects with an increased risk to develop cancer (˜10% in the general population of elderly men according to our present estimates) based on the LOY-status.

A positive result from a CRAY-evaluation could result in a remittance for full oncological evaluation. If these ˜10% with detectable LOY would be remitted for full oncological evaluation, patients with cancer in early and/or late progression could be found. Such detection, and in particular early detection, would generally entail more efficient treatment strategies against the cancer. In this context, it is worth mentioning that 50% median survival in men affected with LOY, i.e. loss of chromosome Y in ≧35% of blood cells, was only 4 years, which should be compared to 11 years average survival in LOY-free men. Hence, 7 years difference in average survival time.

Subjects with somatic LOY and, thus, an increased risk for cancer, but with no detectable cancer after the oncological evaluation, could be recommended for further CRAY-evaluation and oncological evaluation in the future, see FIG. 1. Measurement of LOY in blood could therefore become a useful predictive biomarker of male carcinogenesis.

Hence, a clinical use of the present embodiments is to identify individuals with an increased risk of developing cancer and to target these individuals for more frequent medical examinations.

Further, another clinical use of the present embodiments is to identify suitable treatment regimens for patients at an early state of cancer progression.

The above described applications of CRAY, as well as any other applications of CRAY, could be performed using a kit. Such a kit could be composed of some or all of the following components:
any equipment suitable to detect loss of chromosome Y (LOY) from biological tissue(s);
different approaches or algorithms suitable to analyze the data obtained using the above mentioned equipment and infer the LOY-status from the data;
instructions to relate and compare the inferred LOY-status to pre-defined threshold(s) of risks for cancer; and
instructions and manuals for the use of the kit.

Brief Description of the Study

When studying the impact of post-zygotic genetic aberrations in a large cohort from Uppsala Longitudinal Study of Adult Men (ULSAM) a correlation between somatic loss of chromosome Y (LOY) and risk for non-hematological cancer was discovered. The ULSAM study was initiated in 1970 [17], where 2322 men, born in Uppsala in 1920-1924, participated at the age of 50. The study is investigating a wide range of clinical phenotypes, including cardiovascular diseases, cognitive deficits and dementia as well as cancer history from the National Cancer Registry and the Swedish Civil Registry. Major re-examinations and sampling have been made at ages 60, 70, 77, 82 and 88 years.

According to the embodiments, peripheral blood DNA from 1153 ULSAM participants was genotyped using high-resolution 2.5MHumanOmni SNP-beadchip from Illumina. The population-based ULSAM cohort has extensive phenotypic information of naturally aging men that were clinically followed for >40 years. We studied DNA sampled at an age window of 70.7-83.6 years. Scoring of structural genetic variants was focused on post-zygotic, acquired changes, such as deletions, copy number neutral loss of heterozygozity (CNNLOH), also called acquired uniparental disomy (aUPD), and gains, as described previously [1, 18]. In these analyses 551 autosomal structural variants were uncovered, including 70 deletions, 16 CNNLOH and 465 gains.

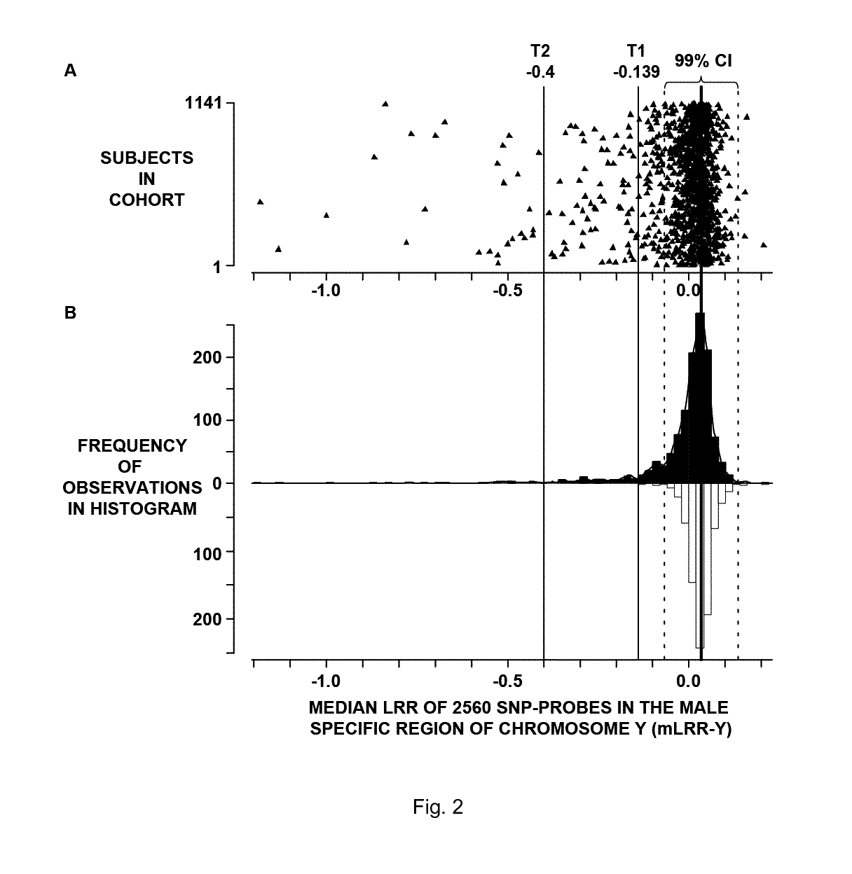

Strikingly, the most frequent somatic variant was the loss of chromosome Y (LOY), see FIG. 2. In brief, the degree of LOY was calculated for each subject and suggested considerable inter-individual differences regarding the proportion of cells with nullisomy Y. A conservative estimate of the frequency of LOY in the cohort was at least 8.2% (see below). At this threshold, >18% of cells in affected participants would be expected to have nullisomy Y. The effects from the above described structural variants on all-cause mortality, cancer mortality and non-cancer-related mortality were examined by Cox proportional hazards regression. In survival analyses, 982 participants free from cancer diagnosis prior to sampling were studied. These tests revealed that LOY was by far the most important risk factor for cancer mortality, including non-hematological cancer mortality (see below) and showed that median survival (50% probability of survival) in the group of men with LOY was 7 years shorter, compared to controls.

Thus, the human chromosome Y is important for biological processes beyond sex determination and reproduction. It has been known for half a century that elderly males frequently lose chromosome Y in normal hematopoietic cells [2, 3]. The clinical consequences of this aneuploidy have been unclear and the prevailing consensus suggests that this mutation should be considered phenotypically neutral and related to normal aging [4-9]. Our results suggest otherwise and clearly point out that LOY as well as other acquired chromosomal abnormalities are associated with negative phenotypic consequences for the affected men.

Frequency of Men with LOY in the Population

We found that somatic loss of chromosome Y (LOY) was the most common structural genetic aberration in the studied ULSAM-cohort of 1153 elderly men, see FIG. 2. An ultra-conservative estimate of the LOY-frequency in this cohort yields that 8.2% are affected as shown in FIG. 2. However, using a 99% confidence interval for estimating the LOY-frequency in the cohort, we see that 14.7% are affected, see FIG. 2. Both of these two mentioned frequencies are very high compared to other known somatic mutations in the general population.

Frequency of Cells with LOY within Subjects

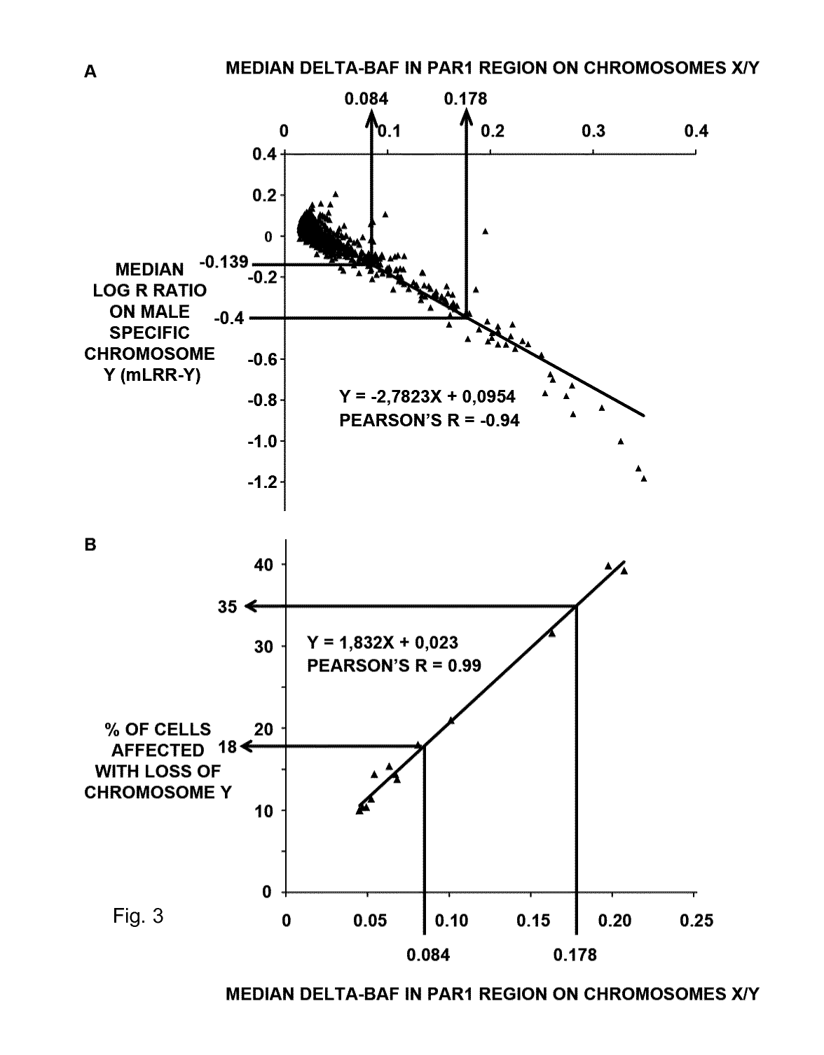

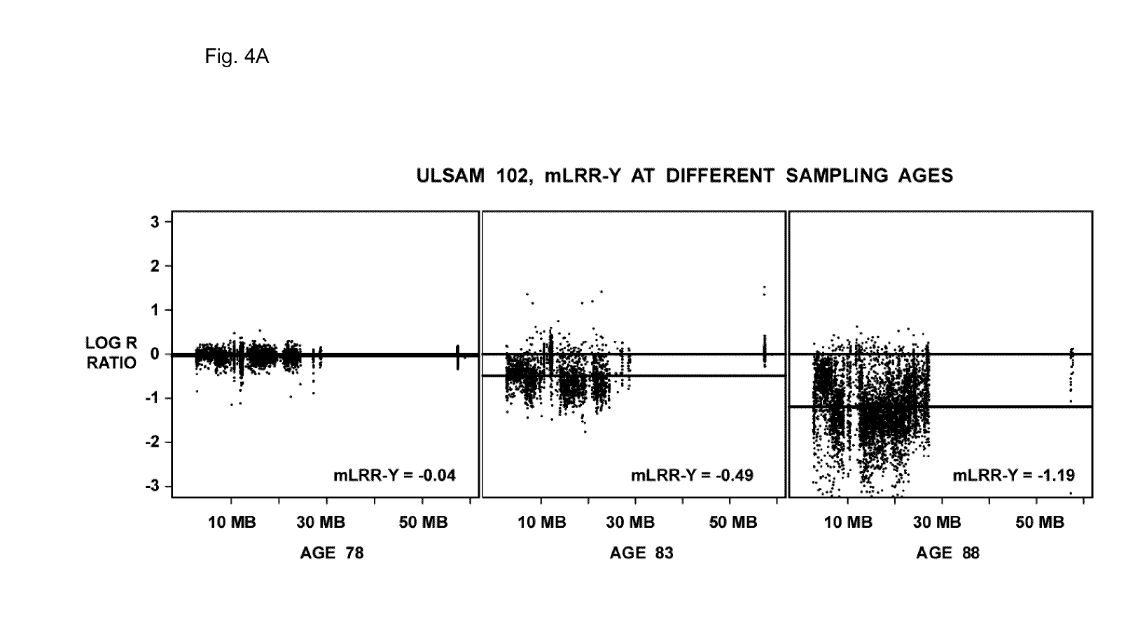

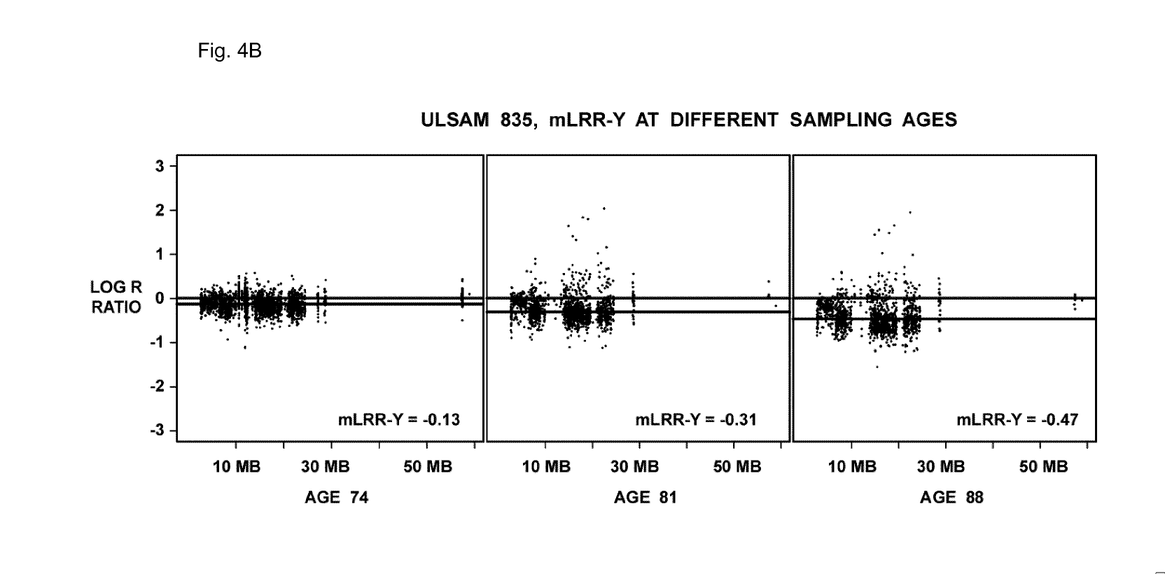

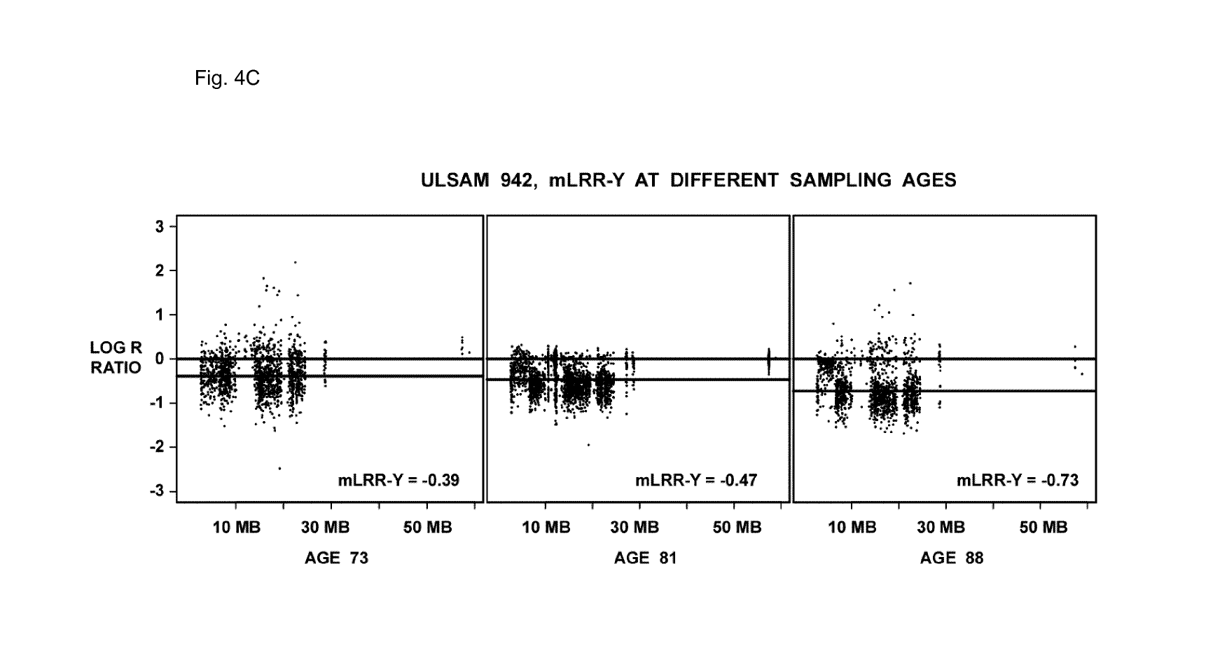

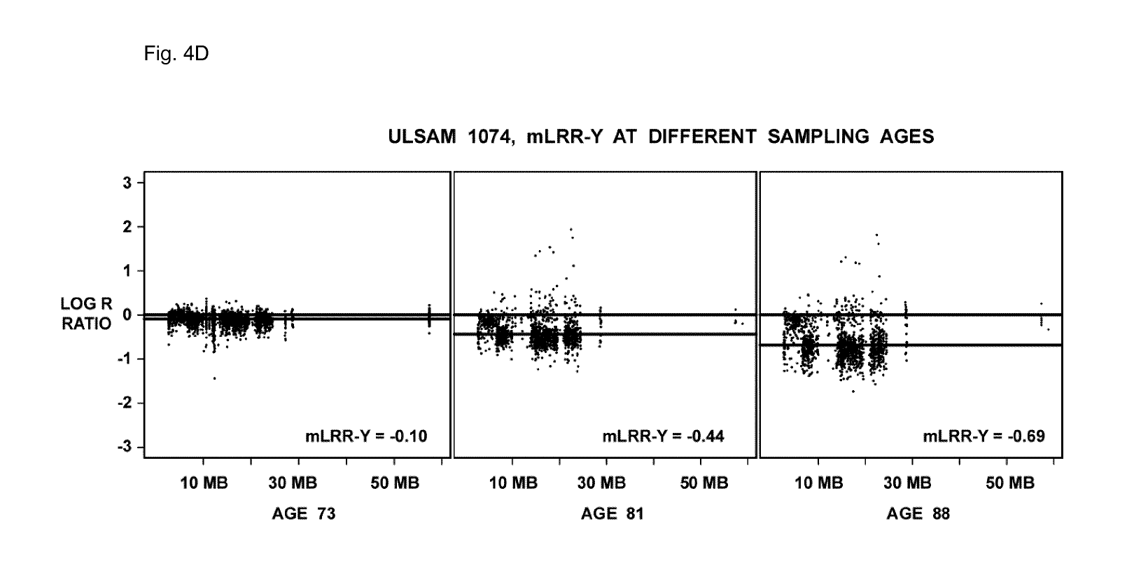

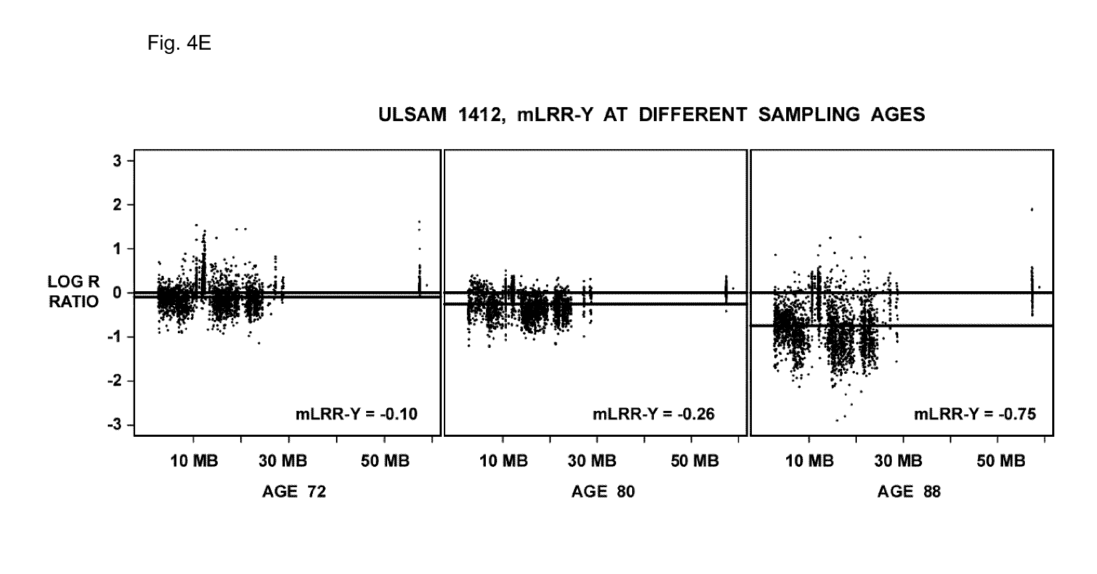

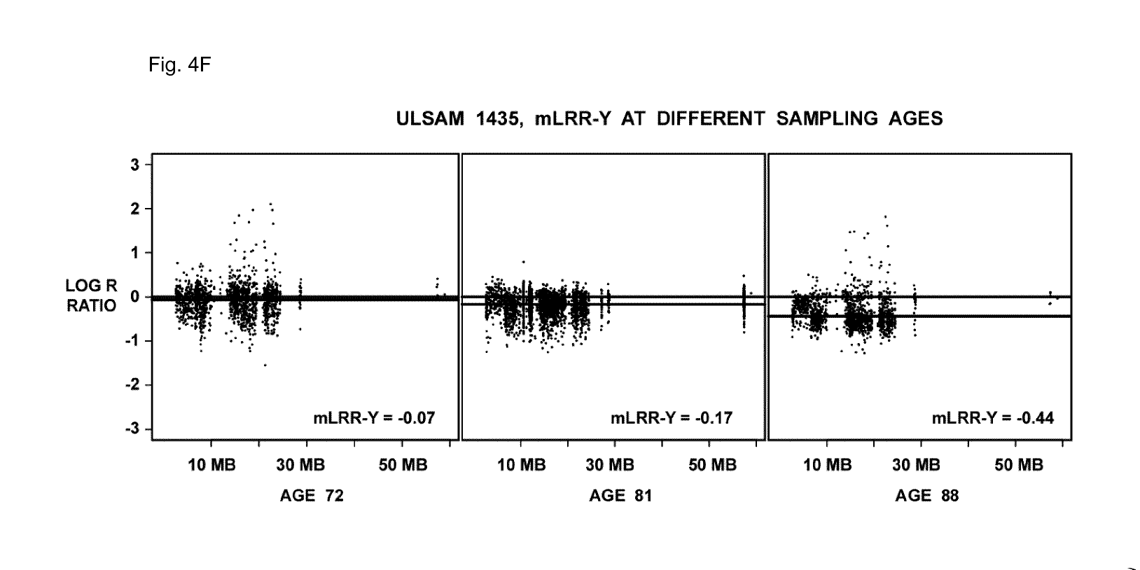

The frequency, or fraction, percentage, degree, of cells with loss of chromosome Y (LOY) was estimated in every subject prior to further analyses and should not be confused with the frequency of men with LOY in the studied population described above. An estimation of the percentage of blood cells affected with LOY was performed through analysis of SNP-array data from the pseudoautosomal region 1 (PAR1) of chromosomes X/Y using MAD-software [16], see FIG. 3. PAR1 is the largest of the PARs, i.e. regions with homologous sequences on chromosomes X and Y, with coordinates 10001-2649520 on Y and 60001-2699520 on X. MAD-software is a tool for detection and quantification of somatic structural variants from SNP-array data, which uses diploid B-allele frequency (BAF) for identification and Log R Ratio (LRR) for quantification of somatic variants and is not originally intended for analyses of chromosome Y data. By using the correlation between the LRR in the PAR1-region of Y and the delta-BAF, i.e. the absolute deviation from the expected BAF-value of 0.5 in heterozygous probes, of the PAR1-region of X/Y, see FIG. 3A, the MAD-quantification of the diploid PAR1 region on chromosomes X/Y could be used to calculate the percentage of cells affected with LOY, see FIG. 3B. Furthermore, the level of LOY, i.e. percentage of affected cells, is not a static, binary feature but is rather changing dynamically over time, see FIG. 4. Through analysis of unique longitudinal samples we described a pattern, in which the number of affected cells is increasing with time within subjects. These observations suggest that the cells affected with LOY are having a proliferative advantage compared to wildtype cells.

Correlations Between LOY, Mortality and Cancer

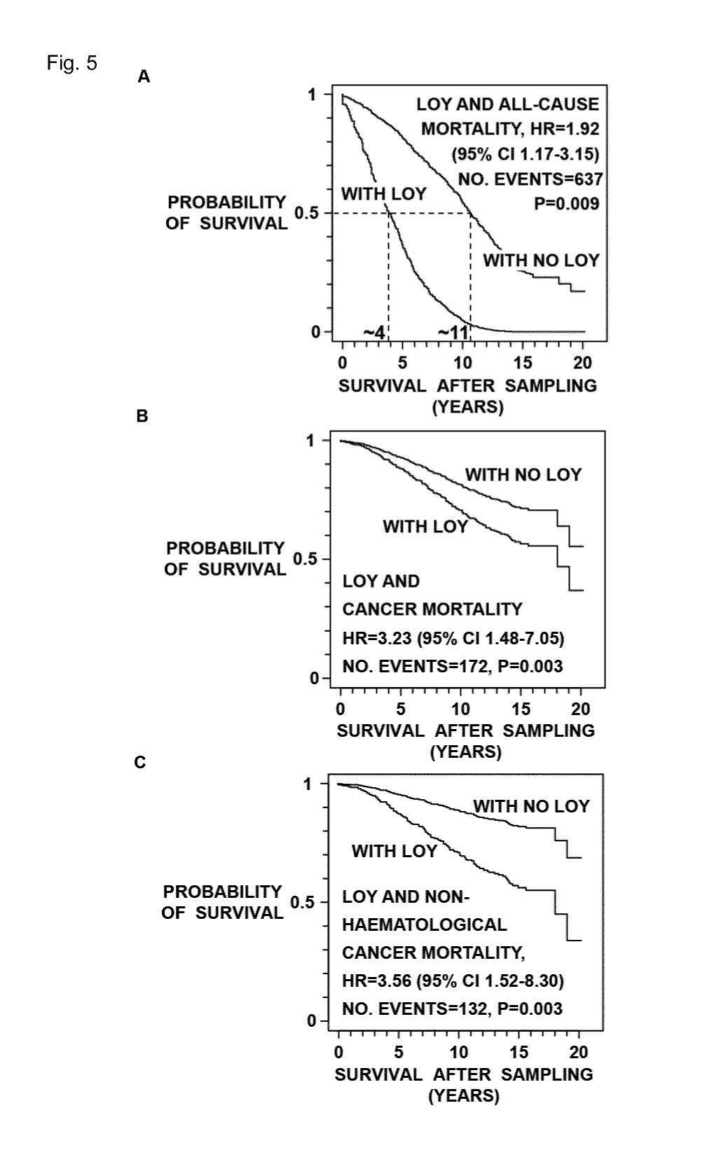

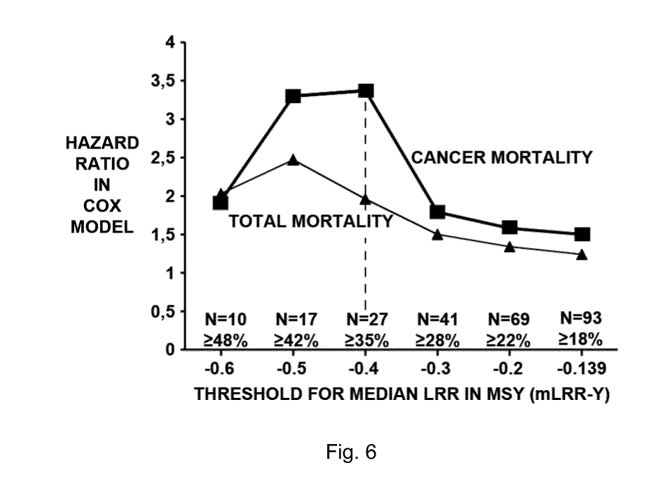

The incidence and mortality for sex-unspecific cancers is higher among men compare to women, a so far largely unexplained difference [19, 20]. Furthermore, age-related loss of chromosome Y (LOY) has been known to occur for decades in normal hematopoietic cells [2, 3] but the phenotypic consequences of LOY have been elusive [4-9]. We have shown herein that somatic loss of chromosome Y (LOY) in the blood cells from men were correlated with reduced survival. Median 50% survival among men in the group affected with LOY was ˜4 years whereas men in the group without detectable LOY had a 50% median survival of ˜11 years, see FIG. 5. Hence, the 50% median survival was 7 years shorter in LOY-men. Specifically, LOY was associated with risks of all-cause mortality (hazard ratio (HR)=1.92, 95% confidence interval (CI)=1.17-3.15, events=637), cancer mortality (HR=3.23, CI=1.48-7.05, events=172) and non-hematological cancer mortality (HR=3.56, CI=1.52-8.30, events=132). The survival analyses were performed using Cox proportional hazards regression and fitted the effects from nine confounding factors as well as genetic factors including LOY, see Table 1. Hence, the CRAY-evaluation could predict the risk for cancer affecting organs outside of the hematological system from DNA sampled from blood cells. As illustrated in FIG. 6, a higher degree of LOY within subjects also seemed to entail larger risks. These results could explain why males are more frequently affected by sex-unspecific cancers. Measurement of LOY in blood can therefore be a useful predictive biomarker of male carcinogenesis.

TABLE 1
Cox proportional hazards regression evaluating effects from LOY on mortality

| | All-cause mortality | | | Cancer mortality | | |
|---|---|---|---|---|---|---|
| | (no of events = 637) | | | (no of events = 172) | | |
| | HR | 95% CI | P-value | HR | 95% CI | P-value |
| Genotyping age | 1.09 | 1.06-1.12 | <0.0001* | 1.06 | 1.00-1.12 | 0.036* |
| Hypertension | 1.66 | 1.34-2.07 | <0.0001* | 1.32 | 0.89-1.96 | 0.167 |
| Exercise habits | 0.49 | 0.32-0.74 | <0.0001* | 1.14 | 0.36-3.59 | 0.827 |
| Smoking | 1.40 | 1.15-1.70 | <0.0001* | 1.36 | 0.93-1.99 | 0.110 |
| Diabetes | 1.41 | 1.08-1.85 | 0.011* | 1.03 | 0.57-1.84 | 0.926 |
| Body mass index (BMI) | 0.99 | 0.96-1.01 | 0.348 | 1.01 | 0.95-1.07 | 0.754 |
| LDL-cholesterol | 0.98 | 0.88-1.07 | 0.504 | 0.95 | 0.79-1.16 | 0.628 |
| HDL-cholesterol | 0.91 | 0.70-1.18 | 0.470 | 0.64 | 0.38-1.09 | 0.099 |
| Education level | 0.94 | 0.87-1.02 | 0.121 | 0.97 | 0.84-1.12 | 0.642 |
| Autosomal Gain | 0.90 | 0.75-1.08 | 0.256 | 0.66 | 0.45-0.95 | 0.027* |
| Autosomal LOH | 1.68 | 1.17-2.41 | 0.005* | 0.64 | 0.24-1.76 | 0.390 |
| LOY | 2.19 | 1.11-4.33 | 0.024* | 3.94 | 1.23-12.56 | 0.021* |

Notes: HR—hazard ratio. 95% CI—95% confidence interval. Autosomal LOH—autosomal loss of heterozygozity. This category was composed of deletions and CNNLOH events. The median follow-up time was 8.7 years (range 0-20.2 years). A continuous explanatory variable was used as a proxy for loss of chromosome Y (mLRR-Y). None of the participants included in analyses (n=982) had any history of cancer before sampling. *—indicates statistically significant effects with 0.05 alpha value.

Out of the 982 participants free from cancer diagnosis prior to sampling, 80 subjects with LOY scored at the level of ≧18% of LOY cells were identified. Of these 80 subjects, 30 later developed cancer according to the distribution of 12 classes of cancer diagnoses as shown in Table 2. For each cancer diagnosis category, the percentage of cases is shown, followed by the absolute number of patients with this diagnosis.

TABLE 2
distribution of cancer diagnoses

| Cancer diagnosis category | % of cases | No. of patients |
|---|---|---|
| Prostate cancer | 6.3% | 5/80 |
| Colorectal cancer | 2.5% | 2/80 |
| Urogenital cancer* | 2.5% | 2/80 |
| Blood and lymphatic cancer | 2.5% | 2/80 |
| Gastrointestinal cancer** | 2.5% | 2/80 |
| Skin cancer*** | 5.0% | 4/80 |
| Lung cancer | 3.8% | 3/80 |
| Ear/Nose/Throat cancer | 1.3% | 1/80 |
| Pancreas cancer | 0% | 0/80 |
| Malignant melanoma | 0% | 0/80 |
| Central nervous system cancer | 5.0% | 4/80 |
| Other cancer | 6.3% | 5/80 |

*excluding prostate cancer
**excluding colorectal and pancreas cancer
***excluding melanoma

Assessment of LOY-Status

After the genetic analyses, or alternative detection method, the loss of chromosome Y-status is assessed from the data produced before the CRAY-evaluation, see FIG. 1. The LOY-status is a proxy for degree of mosaicism, i.e. the percentage of cells, in the analyzed tissue that has lost the Y chromosome. Such LOY-status can be estimated from median Log R Ratio on chromosome Y (mLRR-Y). The Log R Ratio is the type of data from the Illumina SNP-array analysis that shows the copy-number status. The mLRR-Y for each subject is calculated as the median LRR in the male specific region of chromosome Y, between PAR1 and PAR2. Estimations of the percentages of cells that are affected by loss of chromosome Y (LOY) may be obtained by SNP-array and the estimation of percentage can be performed using MAD-software [15]. The exact method for estimating the percentage of cells carrying the aberrations is, however, not of importance for the present embodiments. Any other software or algorithms could also suffice to produce an estimation of degree of mosaicism that will later be used to perform the assessment of LOY-status and risk for cancer.

Thresholds in the Degree of LOY for the CRAY-Evaluation

For the CRAY-evaluation two thresholds in mLRR-Y, T1 and T2, were defined. The first threshold (T1, line at mLRR-Y=−0.139 in FIG. 2) was set at the level of mosaicism where the percentage of cells affected by loss of chromosome Y was clearly not within the expected experimental variation. At this level 18% or more of the examined cells within a sample was affected with LOY. The second threshold (T2, line at mLRR-Y=−0.4 in FIG. 2) was set at the level of mosaicism where Cox survival analyses were giving the most robust regression results, see FIG. 6. In analyses performed in datasets with T2 set at a lower level of mosaicism, such as mLRR-Y=−0.3 or mLRR-Y=−0.2, the low level of mosaicism was giving a smaller biological effect and, thus, lower hazard ratios, see FIG. 6. However, survival analyses performed in datasets with T2 set at a higher level of mosaicism, such as mLRR-Y=−0.5 or mLRR-Y=−0.6, had less statistical power due to few observations in the current dataset to discriminate the effect from LOY on survival and cancer, see FIG. 6. At mLRR-Y=−0.4, 35% or more of the examined cells within a sample was affected with LOY. It is appreciated that the thresholds defined above are based on the data presently available and a person skilled in the art would understand the potential need to adjust the thresholds depending on the results of analyses performed in larger cohorts.

Prediction of Risk with CRAY

CRAY-evaluation of cancer risk was performed by comparing individual degree of somatic loss of chromosome Y to pre-defined risk threshold values, T1 and T2. Subjects with detected somatic LOY where characterized as having increased risk of cancer if mLRR-Y<T1. Our results indicated that subjects in this group were at an increased risk to develop non-hematological cancers as compared to subjects with mLRR-Y>T1. Subjects with even higher degree of somatic LOY were characterized as having great risk of cancer if mLRR-Y<T2. Our results showed that subjects in this group are at a significantly higher risk to develop non-hematological cancers as compared to subjects in which mLRR-Y>T2. see FIG. 5, HR=3.56, CI=1.52-8.30, p=0.003.

In an embodiment, as illustrated in FIG. 1, subjects with mLRR-Y<T1 could be remitted for oncological evaluation. The thresholds were defined from the empirical data of loss of chromosome Y (LOY) and risk for different types of cancer, but they are not static. Analyses indicated that optimal values for the T1 and T2 thresholds could be at −0.139 and −0.4, respectively, as measured in mLRR-Y. Analyses of larger cohorts and using other techniques or methods for LOY-status assessment, may lead to a gradual fine-tuning of optimal values for the T1 and T2 thresholds. An embodiment is directed to the prediction of cancer risk based on individual degree of loss of chromosome Y (LOY) in relation to pre-defined threshold(s), irrespective of the exact value of the threshold(s).

Tissue Analyzed

The correlation between somatic loss of chromosome Y (LOY) and the risk for non-hematological cancer were based on analyses performed using DNA extracted from samples of whole peripheral blood in elderly men. However, any other tissue source of DNA, taken at any age, could be applicable for the CRAY-evaluation.

Methods for Detection of LOY

In an embodiment, genetic experiments performed using SNP-array technology were used. However, the present embodiments are not limited to any particular detection method. Any suitable method or technology could also suffice to produce the data useful to perform the cancer-risk-assessment from LOY-status. In certain subjects it may become useful to repeat the initial genetic experiments and/or to perform secondary or validation experiments before the CRAY-evaluation.

### EXAMPLES

Four examples are presented in which the use and potential benefits of CRAY-evaluations are illustrated. In these examples a CRAY-evaluation could have had a great impact in the treatment of cancers predicted in patients with somatic loss of chromosome Y (LOY). Examples 1-3 are based on the real data with medical history and genotyping data from three longitudinally monitored ULSAM participants, who died from cancer. Their treatment would have been aided by an early LOY-detection and CRAY-evaluation. In the last example, the benefits of CRAY-evaluation are illustrated with a hypothetical cancer patient using two different scenarios. The first of these two scenarios is similar to examples 1-3, since the patient has a detectable risk from LOY-status but still later dies from cancer. In the second scenario, the cancer risk is detected through LOY-status assessment and CRAY-evaluation. The subsequent oncological evaluation diagnosed the patient and the cancer could have been treated in a very early phase of carcinogenesis. Further, in this fictional example, the frequency of defective LOY-cells could be reduced using various treatment alternatives, such as stem cell therapeutics.

### Example 1

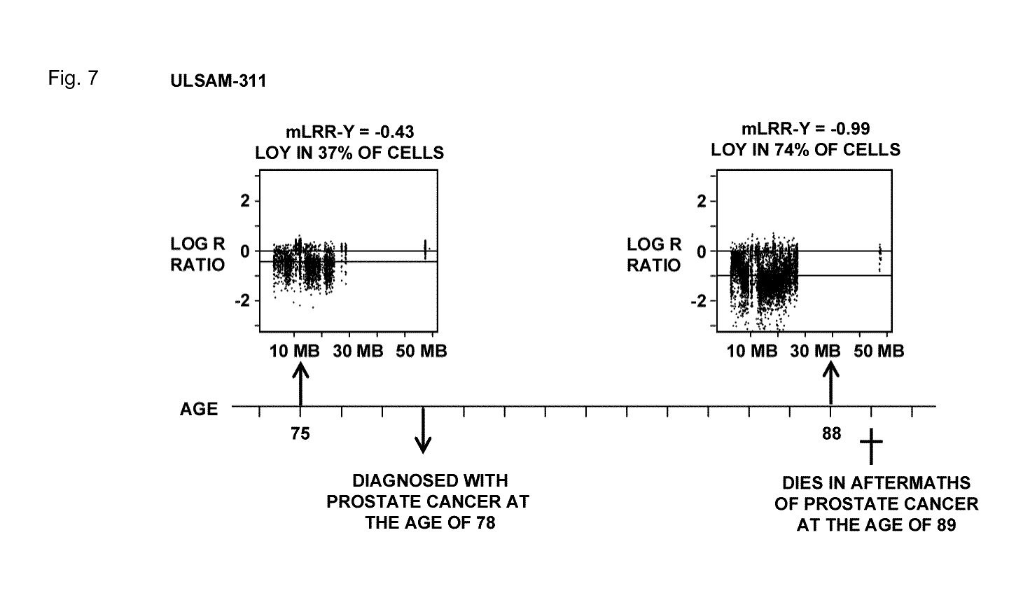

A patient called ULSAM-311 was genotyped on SNP-array at two different ages, 75 and 88 years old. He subsequently died from prostate cancer at the age of 89, see FIG. 7. Somatic loss of chromosome Y (LOY) was present at both genotyping ages with 37% and 75% of cells affected, respectively. A CRAY-evaluation at both of these ages would have predicted a great risk of cancer. For example, an oncological evaluation at 75 as initiated by a CRAY-evaluation performed at the 75 years DNA could have resulted in a three years earlier diagnosis of the prostate cancer that subsequently led to the death of this man. Screening of the blood at younger ages would probably entail an even earlier diagnosis. See FIG. 7.

### Example 2

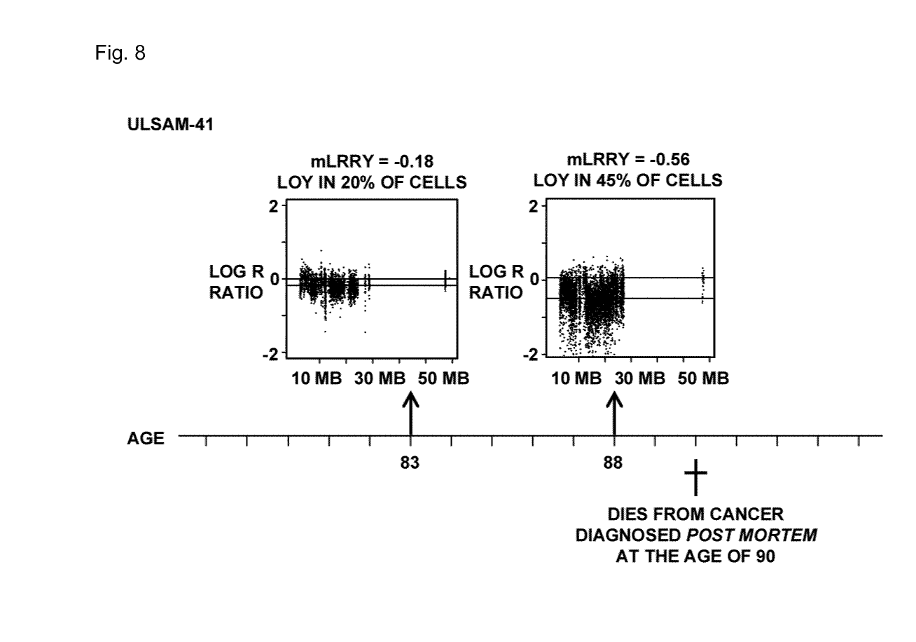

A subject ULSAM-41 was genotyped using SNP-array at two ages, 83 and 88 years old, and died from an un-diagnosed cancer at the age of 90, see FIG. 8. Somatic loss of chromosome Y (LOY) was present at both genotyping ages with 20% and 45% of cells affected, respectively. A CRAY-evaluation at any of these ages would have predicted an increased risk of cancer and subsequently a great risk of cancer. The following oncological examination could have detected the cancer that caused the death of this man seven years later. For example, a CRAY-evaluation using the data from the sample collected at 83 years of age would have predicted an increased risk for cancer and a better treatment for this man. However, the cancer that caused the mortality was only discovered post mortem. See FIG. 8.

### Example 3

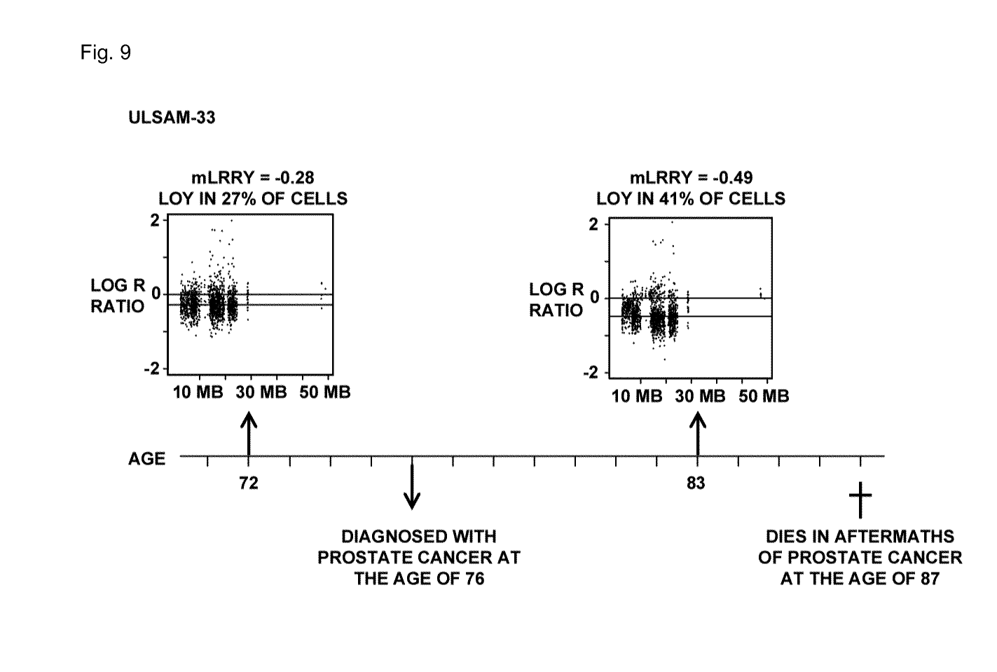

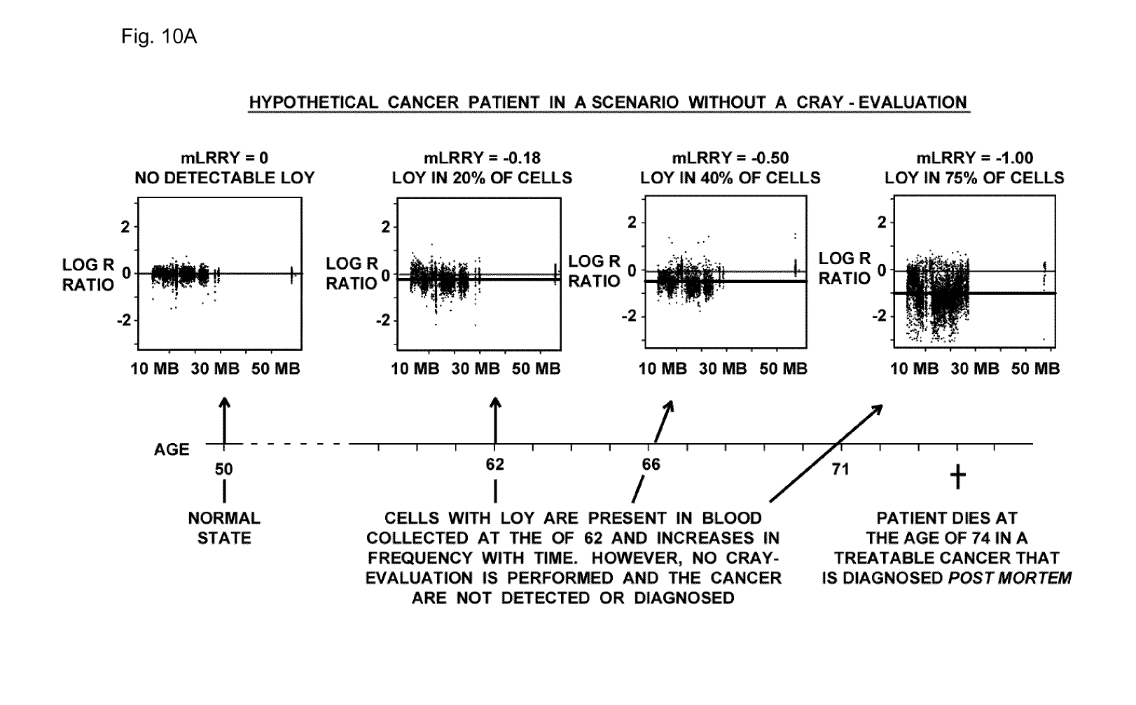

A participant ULSAM-33 was genotyped on SNP-array at two ages, 72 and 83 years old, and died from prostate cancer at the age of 87, see FIG. 9. Somatic loss of chromosome Y (LOY) was present at both genotyping ages with 27% and 41% of cells affected, respectively. A CRAY-evaluation at both of these ages would have predicted an increased cancer risk and subsequently a great risk of cancer. For example, an oncological evaluation at 72 as initiated by a CRAY-evaluation performed at the 72 years DNA could have resulted in a four years earlier diagnosis of the prostate cancer that subsequently led to his death. Screening of blood sampled at even younger ages would probably entail an even earlier diagnosis. See FIG. 9.

### Example 4

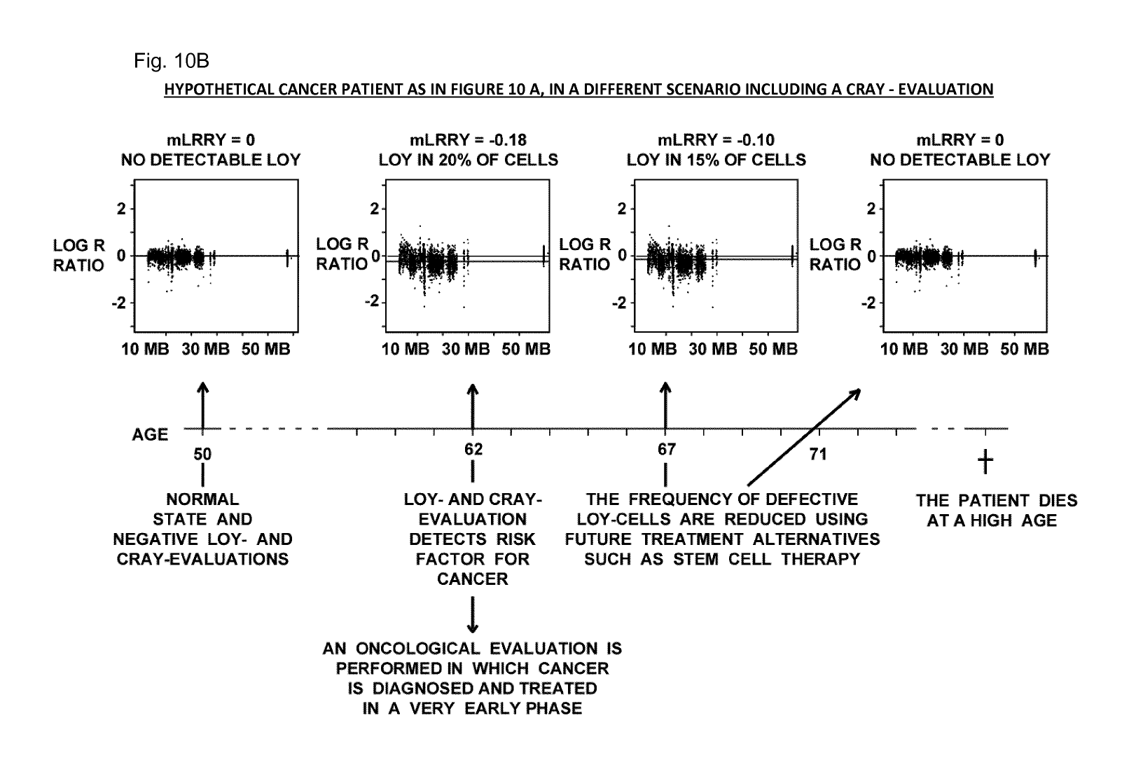

This example illustrates the benefits of CRAY-evaluation using a hypothetical cancer patient in two different scenarios, see FIG. 10. In the first scenario, no CRAY-evaluation is performed and the subject dies from a cancer at the age of 75. This scenario is very similar to examples 1-3 and the clinical reality of contemporary cancer screening. In the alternative scenario, the CRAY-evaluation performed at the age of 65 detects the risk factor for cancer, i.e. LOY. In this patient an oncological evaluation could diagnose a cancer that was treated in a very early phase of carcinogenesis. Furthermore, we anticipate that the frequency of defective LOY-cells can be reduced using a variety of future treatment strategies, such as stem cell therapy or adjuvant stimulation. See FIG. 10.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

[1] Forsberg, L. A., Absher, D. & Dumanski, J. P. Non-heritable genetics of human disease: spotlight on post-zygotic genetic variation acquired during lifetime. Journal of medical genetics 50, 1-10 (2013).
[2] Jacobs, P. A., Brunton, M., Court Brown, W. M., Doll, R. & Goldstein, H. Change of human chromosome count distribution with age: evidence for a sex differences. Nature 197, 1080-1 (1963).
[3] Pierre, R. V. & Hoagland, H. C. Age-associated aneuploidy: loss of Y chromosome from human bone marrow cells with aging. Cancer 30, 889-94 (1972).
[4] Nowinski, G. P. et al. The frequency of aneuploidy in cultured lymphocytes is correlated with age and gender but not with reproductive history. Am J Hum Genet 46, 1101-11 (1990).
[5] Loss of the Y chromosome from normal and neoplastic bone marrows. United Kingdom Cancer Cytogenetics Group (UKCCG). Genes, chromosomes cancer 5, 83-8 (1992).
[6] Wiktor, A. et al. Clinical significance of Y chromosome loss in hematologic disease. Genes, chromosomes & cancer 27, 11-6 (2000).
[7] Wong, A. K. et al. Loss of the Y chromosome: an age-related or clonal phenomenon in acute myelogenous leukemia/myelodysplastic syndrome? Archives of pathology & laboratory medicine 132, 1329-32 (2008).
[8] Wiktor, A. E., Van Dyke, D. L., Hodnefield, J. M., Eckel-Passow, J. & Hanson, C. A. The significance of isolated Y chromosome loss in bone marrow metaphase cells from males over age 50 years. Leukemia research 35, 1297-300 (2011).
[9] Jacobs, P. A. et al. Male breast cancer, age and sex chromosome aneuploidy. Br J Cancer 108, 959-63 (2013).
[10] Hunter, S., Gramlich, T., Abbott, K. & Varma, V. Y chromosome loss in esophageal carcinoma: an in situ hybridization study. Genes, chromosomes & cancer 8, 172-7 (1993).
[11] Vijayakumar, S. et al. Detection of recurrent copy number loss at Yp11.2 involving TSPY gene cluster in prostate cancer using array-based comparative genomic hybridization. Cancer Res 66, 4055-64 (2006).
[12] Zhang, L. J., Shin, E. S., Yu, Z. X. & Li, S. B. Molecular genetic evidence of Y chromosome loss in male patients with hematological disorders. Chin Med J (Engl) 120, 2002-5 (2007).
[13] Bianchi, N. O. Y chromosome structural and functional changes in human malignant diseases. Mutation research 682, 21-7 (2009).
[14] Veiga, L. C. S., Bergamo, N. A., Reis, P. P., Kowalski, L. P. & Rogatto, S. R. Loss of Y-chromosome does not correlate with age at onset of head and neck carcinoma: a case-control study. Brazilian Journal of Medical and Biological Research 45, 172-178 (2012).
[15] Pique-Regi, R., Caceres, A. & Gonzalez, J. R. R-Gada: a fast and flexible pipeline for copy number analysis in association studies. BMC Bioinformatics 11, 380 (2010).
[16] Gonzalez, J. R. et al. A fast and accurate method to detect allelic genomic imbalances underlying mosaic rearrangements using SNP array data. BMC bioinformatics 12, 166 (2011).
[17] Hedstrand, H. A study of middle-aged men with particular reference to risk factors for cardiovascular disease. Ups J Med Sci Suppl 19, 1-61 (1975).
[18] Forsberg, L. et al. Age-related somatic structural changes in the nuclear genome of human blood cells. Am J Hum Genet 90, 217-28 (2012).
[19] Cook, M. B., McGlynn, K. A., Devesa, S. S., Freedman, N. D. & Anderson, W. F. Sex disparities in cancer mortality and survival. Cancer epidemiology, biomarkers & prevention: a publication of the American Association for Cancer Research, cosponsored by the American Society of Preventive Oncology 20, 1629-37 (2011).
[20] Edgren, G., Liang, L., Adami, H. O. & Chang, E. T. Enigmatic sex disparities in cancer incidence. European journal of epidemiology 27, 187-96 (2012).

1. A method of predicting whether a male subject has an increased risk of developing cancer, comprising:
determining (S1) a fraction of cells from a biological sample obtained from said male subject that have lost chromosome Y;
comparing (S2) said fraction to a predefined threshold; and
predicting (S3) whether said male subject has an increased risk of developing cancer based on said comparison between said fraction and said predefined threshold.

2. The method according to claim 1, wherein determining (S1) said fraction comprises determining (S1) a fraction of somatic cells from said biological sample that have lost chromosome Y.

3. The method according to claim 1 or 2, wherein predicting (S3) whether said male subject has an increased risk of developing cancer comprises predicting (S3) whether said male subject has an increased risk of developing a non-hematological cancer based on said comparison between said fraction and said predefined threshold.

4. The method according to any of claims 1 to 3, wherein the cells in step S1 are nucleated blood cells from a blood sample obtained (from said male subject, and determining (S1) said fraction comprises determining (S1) a fraction of nucleated blood cells from said blood sample that have lost chromosome Y.

5. The method according to any one of claims 1 to 4, further comprising obtaining a subset or fraction of cells from the biological sample and determining (S1) a fraction of cells from said subset or fraction that have lost chromosome Y.

6. The method according to any one of claims 1 to 5, wherein determining (S1) said fraction comprises determining a fraction of CD4+ T-cells that have lost chromosome Y.

7. The method according to any of claims 1 to 6, wherein predicting (S3) whether said male subject has an increased risk of developing cancer comprises:
predicting that said male subject has an increased risk of developing cancer if said fraction is equal to or higher than said predefined threshold; and
predicting that said male subject does not have any increased risk of developing cancer if said fraction is lower than said predefined threshold.

8. The method according to any one of claims 1 to 7, wherein a said predefined threshold lies in the range 5 to 20% of cells.

9. The method according to any of claims 1 to 8, wherein comparing (S2) said fraction comprises comparing (S2) said fraction to a predefined threshold of about 18%.

10. The method according to any of claims 1 to 8, wherein a predefined threshold is set at the lower 99% confidence interval of the distribution of expected experimental background noise in a determination of frequency of loss of chromosome Y.

11. The method according to any of the claims 1 to 10, wherein
comparing (S2) said fraction comprises comparing (S2) said fraction to a first predefined threshold and a second predefined threshold that is higher than said first predefined threshold, and
predicting (S3) whether said male subject has an increased of developing cancer comprises:
predicting that said male subject does not have any increased risk of developing cancer if said fraction is lower than said first predefined threshold;
predicting that said male subject has an increased risk of developing cancer if said fraction is equal to or larger than said first predefined threshold but lower than said second predefined threshold; and
predicting that said male subject has a high risk of developing cancer if said fraction is equal to or larger than said second predefined threshold.

12. The method according to claim 11, wherein comparing (S2) said fraction comprises comparing (S2) said fraction to said first predefined threshold of about 18% and said second predefined threshold of about 35%.

13. The method according to any of the claims 1 to 12, wherein determining (S1) said fraction comprises determining (S1) said fraction based on a single-nucleotide polymorphism, SNP, array analysis using data points derived from a pseudoautosomal region 1, PAR1, region of chromosomes X and Y.

14. The method according to claim 13, wherein determining (S1) said fraction comprises determining (S1) said fraction based on a quantification of said data points derived from said PAR1 region on chromosomes X and Y by using a correlation between a Log R Ratio, LRR, in said PAR1 region of chromosome Y and an absolute deviation from an expected diploid B-allele frequency, BAF, value of 0.5.

15. The method according to any of the claims 1 to 12, wherein determining (S1) said fraction comprises determining (S1) said fraction based on sequencing of a DNA region present on chromosome Y.

16. The method according to any of the claims 1 to 15 wherein the subject is healthy, not suffering from cancer or not identified as having cancer.

17. A kit for predicting whether a male subject has an increased risk of developing cancer, said kit comprises:
means for determining a fraction of cells from a biological sample obtained from said male subject that have lost chromosome Y;
instructions for comparing said fraction to a threshold defined in said instructions; and
instructions for predicting whether said male subject has an increased risk of developing cancer based on said comparison between said fraction and said threshold.

18. Use of loss of chromosome Y in cells from a biological sample obtained from a male subject as a genetic marker to predict whether said male subject has an increased risk of developing cancer.

19. A method of investigating the association between cancer, or the risk of developing cancer, said method comprising determining the fraction of cells in a sample which have lost chromosome Y.

20. The method of claim 19 wherein the sample comprises cells obtained or derived from a subject which has previously undergone a risk prediction assessment according to the method of any one of claims 1 to 16.
